(19) United States
(12) Patent Application Publication
(10) Pub. No.: US 2017/0266289 A1
(43) Pub. Date: Sep. 21, 2017

(54) TOPICAL FORMULATION

(71) Applicant: AbbVie Inc., North Chicago, IL (US)

(72) Inventors: John M. Lipari, Mt. Pleasant, WI (US); Philip D. Zocharski, Chelsea, MI (US)

(73) Assignee: AbbVie Inc., North Chicago, IL (US)

(21) Appl. No.: 15/506,310

(22) PCT Filed: Aug. 27, 2015
(86) PCT No.: PCT/US2015/047152

§ 371 Date: Feb. 24, 2017

Related U.S. Application Data
(60) Provisional application No. 62/042,600, filed on Aug. 27, 2014.

(51) Int. Cl. A61K 47/22; A61K 47/26; A61K 47/32; A61Q 19/00; A61K 8/49; A61K 9/06; A61K 9/00; A61K 47/10; A61K 8/81

(52) U.S. Cl. CPC A61K 47/22; A61K 47/10; A61K 47/26; A61K 47/32; A61K 8/8158; A61K 8/4993; A61K 9/06; A61K 9/0014; A61Q 19/00

(57) ABSTRACT

The invention described herein provides topical formulations that can be prepared at ambient temperature without the need for any heating step during preparation. Thus the formulations are particularly suitable for cosmetic and pharmaceutical active ingredients that are relatively heat sensitive. The invention also provides methods for preparing the same.

### REFERENCE TO RELATED APPLICATION

This application claims the benefit of the filing date under 35 U.S.C. 119(e) to U.S. Provisional Application No. 62/042,600, filed on Aug. 27, 2014, the entire content of which is incorporated herein by reference.

### BACKGROUND OF THE INVENTION

Compared to other delivery methods and systems, transdermal or topical drug delivery systems present great advantages, including non-invasiveness, higher concentrations at the site of action, prolonged therapeutic effect, reduced systemic side effects, better patient compliance and easy termination of drug therapy.

Successful delivery of a cosmetic or pharmaceutical composition in a mammal through the topical route, however, relies on the ability of the cosmetic or pharmaceutical composition to penetrate the outer layer of the epidermis known as the stratum corneum (SC). The stratum corneum is comprised mainly of about ten to twenty layers of flattened dead cells (corneocytes) filled with keratin. Lipids, such as free fatty acids, cholesterol, and ceramides, connect the regions between the keratinized cells, forming a brick and mortar-like structure. Diffusion into the skin is mainly influenced by the stratum corneum, hair follicles, and ducts. Underneath the stratum corneum is a viable epidermis layer, a dermis layer, followed by a hypodermis layer, which are vascularized, allowing the transport of substances (e.g., Active Pharmaceutical Ingredient or API) that have managed to cross the stratum corneum into systemic circulation. See FIG. 8. In mammals, this structure primarily serves as a barrier to chemicals and biological agents, including bacteria, fungus, and viruses.

The penetration of cosmetic or pharmaceutical compositions through the stratum corneum occurs primarily through passive transport mechanisms. Passive delivery or diffusion relies on a concentration density gradient between the drug at the outer surface and the inner surface of the skin. The diffusion rate is proportional to the gradient and is modulated by a molecule's size, hydrophobicity, hydrophilicity and other physiochemical properties, as well as the area of the absorptive surface. Examples of passive delivery systems include transdermal patches for controlled delivery of, for example, nitroglycerine (angina), scopolamine (motion sickness), fentanyl (pain control), nicotine (smoking cessation), estrogen (hormone replacement therapy), testosterone (male hypogonadism), clonidine (hypertension), and lidocaine (topical anesthesia). Formulations can be applied directly to the skin or a controlled delivery method may be used. The controlled delivery of these drugs can include the use of polymer matrices, reservoirs containing drugs with rate-controlling membranes and drug-in-adhesive systems.

One issue associated with the preparation of topical formulation is that most such formulations, such as creams, requires a heating process during the preparation of the formulation, thus potentially limiting the range of cosmetic or pharmaceutical active ingredients that can be formulated in such formulations, particularly when active ingredients exhibit chemical sensitivities at elevated temperatures. Thus there exists a need for topical formulations that do not require a heating step for preparation.

### SUMMARY OF THE INVENTION

In a first embodiment the invention provides a method for producing a formulation for topical application to skin or mucosal surface, the method comprising:
(1) mixing an oil phase with an emulsifying agent to form a mixture, and agitating the mixture until homogeneous;
(2) adding an aqueous phase and continuing to agitate to form a placebo formulation;
(3) adding a solid form active ingredient to the placebo formulation, and mixing until homogeneous;

wherein the method does not comprise a step that subjects the solid form active ingredient to a temperature higher than 35° C.

In a second embodiment the invention provides a method according to the first embodiment of the invention, wherein the solid form active ingredient is not subjected to a temperature higher than 15-30° C., or is subjected to a temperature between 15-30° C.

In a third embodiment the invention provides a method according to any of the foregoing embodiments, wherein the emulsifying agent is also a thickener, a stabilizing agent, or both.

In a fourth embodiment the invention provides a method according to any of the foregoing embodiments, wherein the method further comprises independently adding a thickener, a stabilizing agent, or both a thickener and a stabilizing agent, in step (1) and/or step (2). The thickener and stabilizing agent can be added together or separately in step (1) and/or step (2).

In a fifth embodiment the invention provides a method according to any of the foregoing embodiments, wherein the thickener does not require heating, or require heating above 35° C., for use in a topical formulation.

In a sixth embodiment the invention provides a method according to any of the foregoing embodiments, wherein the thickener comprises one or more of: silicon dioxide; xanthan gum; high molecular weight, crosslinked, acrylic acid-based polymers; polyvinyl alcohol; agarose; alginate; carrageenan; guar gum; cellulose derivative; methylcellulose; sodium carboxymethylcellulose; hydroxypropyl cellulose; hydroxypropyl methylcellulose; hydroxyethyl cellulose; and povidone.

In a seventh embodiment the invention provides a method according to any of the foregoing embodiments, wherein the stabilizing agent comprises: lauryl glucoside, decyl glucoside sodium cocoamphoacetate, polyglyceryl 3-methylglucose distearate, cetearyl glucoside, inulin lauryl carbamate, lecithin and its derivatives, purified phospholipids, polysorbate 80, sorbitan monooleate, or mixtures thereof.

In an eighth embodiment the invention provides a method according to any of the foregoing embodiments, wherein the stabilizing agent comprises: polyoxyethylene 20 sorbitan monooleate; polyoxyethylene 20 sorbitan monolaurate; polyoxyethylene 4 sorbitan monolaurate; polyoxyethylene 20 sorbitan monopalmitate; polyoxyethylene 5 sorbitan monooleate; polyoxyethylene 20 sorbitan trioleate; sorbitan monolaurate; sorbitan monooleate; sorbitan trioleate; polyoxyl 35 castor oil; and mixtures thereof.

In a ninth embodiment the invention provides a method according to any of the foregoing embodiments, wherein the stabilizing agent comprises a polyoxyethylene sorbitan fatty acid ester.

In a tenth embodiment the invention provides a method according to any of the foregoing embodiments, wherein the stabilizing agent comprises a polyoxyethylene castor oil derivative.

In an eleventh embodiment the invention provides a method according to any of the foregoing embodiments, wherein the stabilizing agent comprises a pharmaceutically acceptable liquid stabilizer that can be processed at ambient conditions (e.g., 15-30° C.) for use in topical formulation.

In a twelfth embodiment the invention provides a method according to any of the foregoing embodiments, wherein the method further comprises, adding a preservative after step (1). For example, the preservative can be added to the homogeneous mixture after step (1), shortly before, after, or simultaneously with the addition of the aqueous phase in step (2). The preservative may be added to the aqueous phase before step (2) (that is, the aqueous phase used in step (2) comprises a preservative). Alternatively, the method may further comprise adding a preservative to the aqueous phase used in step (2).

In a thirteenth embodiment the invention provides a method according to any of the foregoing embodiments, wherein the method further comprises adding a permeation enhancer to the mixture of step (1), between steps (1) and (2), to the aqueous phase of step (2), or between steps (2) and (3). Alternatively, the aqueous phase used in step (2) comprises a permeation enhancer.

In a fourteenth embodiment the invention provides a method according to any of the foregoing embodiments, wherein the method further comprises measuring droplet particle size periodically during step (2).

In a fifteenth embodiment the invention provides a method according to any of the foregoing embodiments, wherein the formulation is an oil-in-water emulsion, or a water-in-oil emulsion.

In a sixteenth embodiment the invention provides a method according to any of the foregoing embodiments, wherein the formulation is a lotion, a cream, a gel, or a gel-cream.

In a seventeenth embodiment the invention provides a method according to any of the foregoing embodiments, wherein the oil phase is about 1-30% (w/w), about 10-20% (w/w), or about 15% (w/w) of the formulation.

In an eighteenth embodiment the invention provides a method according to any of the foregoing embodiments, wherein the emulsifying agent is about 1-5% (w/w), or about 3% of the formulation.

In a nineteenth embodiment the invention provides a method according to any of the foregoing embodiments, wherein the preservative is about 0.001-2% (w/w), about 0.01-1% (w/w), about 0.1-0.5% (w/w), or about 0.2% of the formulation.

In a twentieth embodiment the invention provides a method according to any of the foregoing embodiments, wherein the permeation enhancer is less than 15% (w/w), or about 1-10% of the formulation.

In a twenty first embodiment the invention provides a method according to any of the foregoing embodiments, wherein the solid form active ingredient is about 0.01-10% (w/w), about 0.05-5% (w/w), about 0.1-1% (w/w), or about 0.5-1% (w/w) of the formulation.

In a twenty second embodiment the invention provides a method according to any of the foregoing embodiments, wherein the oil phase comprises a pharmaceutically acceptable oil that can be processed at ambient conditions for use in topical formulation.

In a twenty third embodiment the invention provides a method according to any of the foregoing embodiments, wherein the oil phase comprises: cod liver oil, light mineral oil, heavy mineral oil, shark liver oil, caprylic/capric triglyceride, vegetable oil, or mixtures thereof.

In a twenty fourth embodiment the invention provides a method according to any of the foregoing embodiments, wherein the vegetable oil comprises: castor oil, corn oil, canola oil, cottonseed oil, peanut oil, sesame oil, or soybean oil.

In a twenty fifth embodiment the invention provides a method according to any of the foregoing embodiments, wherein the emulsifying agent comprises: sodium lauryl sulfate, or a non-ionic emulsifier.

In a twenty sixth embodiment the invention provides a method according to any of the foregoing embodiments, wherein the emulsifying agent comprises a SEPINEO™ P 600-type hydro swelling droplet polymer in an inverse emulsion.

In a twenty seventh embodiment the invention provides a method according to any of the foregoing embodiments, wherein the emulsifying agent is an inverse emulsion and is comprised of acrylamide/sodium acryloyldimethyl taurate copolymer/isohexadecane/polysorbate 80, or SEPINEO™ P 600 brand HSD polymer.

In a twenty eighth embodiment the invention provides a method according to any of the foregoing embodiments, wherein the preservative comprises: sodium benzoate, benzoic acid, sorbic acid, benzethonium chloride, benzalkonium chloride, bronopol, methylparaben, ethylparaben, propylparaben, butylparaben, thiomerosal, sodium propionate, chlorhexidine, chlorobutanol, chlorocresol, cresol, imidazolidinyl urea, diazolidinyl urea, phenol, phenylmercuric salts, potassium sorbate, propylene glycol, or mixtures thereof.

In a twenty ninth embodiment the invention provides a method according to any of the foregoing embodiments, wherein the preservative comprises imidazolidinyl urea.

In a thirtieth embodiment the invention provides a method according to any of the foregoing embodiments, wherein the permeation enhancer comprises: dimethyl isosorbide, isopropyl myristate, diethylene glycol monoethyl ether, ethyl alcohol, ethyl oleate, isostearyl alcohol, oleyl alcohol, polyethylene glycol, N-methyl-2-pyrrolidone, dimethyl sulfoxide, or propylene carbonate.

In a thirty first embodiment the invention provides a method according to any of the foregoing embodiments, wherein the aqueous phase is purified water.

In a thirty second embodiment the invention provides a method according to any of the foregoing embodiments, wherein the solid form active ingredient is a cosmetic or pharmaceutical active ingredient.

In a thirty third embodiment the invention provides a method according to any of the foregoing embodiments, wherein the solid form active ingredient can be any pharmaceutical agent which can be applied topically. For example, the solid form active ingredient can be scopolamine (motion sickness), fentanyl (pain control), nicotine (smoking cessation), estrogen (hormone replacement therapy), testosterone (male hypogonadism), clonidine (hypertension), and lidocaine (topical anesthesia), or can be Compound 1 (N-((1S,3R,4S)-3-ethyl-4-(6H-pyrrolo[2,3-e][1,2,4]triazolo[4,3-a]pyrazin-1-yl)cyclopentyl)cyclopropanesulfonamide), Compound 2 ((3S,4R)-3-ethyl-4-(3H-imidazo[1,2-a]pyrrolo[2,3-e]pyrazin-8-yl)-N-(2,2,2-trifluoroethyl)pyrrolidine-1-carboxamide), cyclosporine A or betamethasone dipropionate; or wherein the solid form active ingredient is effective to treat psoriasis, plaque psoriasis, nail psoriasis, psoriatic arthritis, hidradenitis suppurativa, alopecia areata/universalis, vitiligo, AKT keratosis (age spots), itch, or atopic dermatitis.

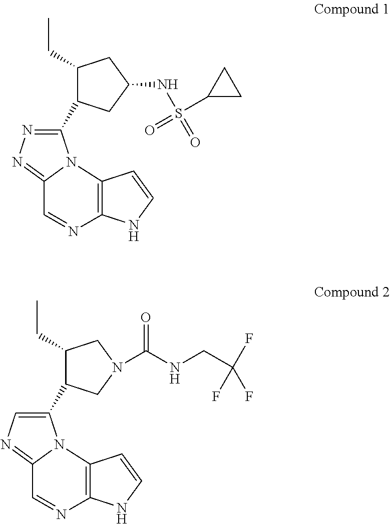

In a thirty fourth embodiment the invention provides a method according to any of the foregoing embodiments, wherein the method further comprising, after Step (1) or Step (2), adding a solid form active ingredient and mixing until homogenous.

In a thirty fifth embodiment the invention provides a cosmetic or pharmaceutical topical formulation for applying to skin or a mucosal surface, said topical formulation comprising:
(1) about 1-30% (w/w) (e.g., about 10-20%, or about 15%) oil (e.g., mineral oil);
(2) about 1-5% (w/w) emulsifying agent, which can be a SEPINEO™ P 600-type hydro swelling droplet (HSD) polymer;
(3) about 1-15% (w/w) permeation enhancer;
(4) about 0.001-2% (w/w) preservative;
(5) about 65-90% (w/w) aqueous phase; and
(6) about 0.01-10% (w/w) cosmetic or pharmaceutical active ingredient.

In a thirty sixth embodiment the invention provides a cosmetic or pharmaceutical topical formulation for applying to skin or a mucosal surface, said topical formulation comprising:
(1) about 10-20% (w/w) oil;
(2) about 1-5% (w/w) emulsifying agent, which can be a SEPINEO™ P 600-type hydro swelling droplet (HSD) polymer (such as acrylamide/sodium acryloyldimethyl taurate copolymer/isohexadecane/polysorbate 80, or SEPINEO™ P 600 brand HSD polymer);
(3) about 1-15% (w/w) permeation enhancer;
(4) about 0.001-2% (w/w) preservative;
(5) about 65-90% (w/w) aqueous phase; and
(6) about 0.01-10% (w/w) cosmetic or pharmaceutical active ingredient.

In a thirty seventh embodiment the invention provides a cosmetic or pharmaceutical topical formulation for applying to skin or a mucosal surface, said topical formulation comprising:
(1) about 10-20% (w/w) mineral oil;
(2) about 1-5% (w/w) SEPINEO™ P 600-type hydro swelling droplet (HSD) polymer (such as acrylamide/sodium acryloyldimethyl taurate copolymer/isohexadecane/polysorbate 80, or SEPINEO™ P 600 brand HSD polymer);
(3) about 1-15% (w/w) permeation enhancer;
(4) about 0.001-2% (w/w) preservative;
(5) about 65-90% (w/w) aqueous phase; and
(6) about 0.01-10% (w/w) cosmetic or pharmaceutical active ingredient.

In a thirty eighth embodiment the invention provides a cosmetic or pharmaceutical topical formulation for applying to skin or a mucosal surface, said topical formulation comprising:
(1) about 10-20% (w/w) mineral oil;
(2) about 3% (w/w) SEPINEO™ P 600-type hydro swelling droplet (HSD) polymer (such as acrylamide/sodium acryloyldimethyl taurate copolymer/isohexadecane/polysorbate 80, or SEPINEO™ P 600 brand HSD polymer);
(3) about 1-15% (w/w) permeation enhancer;
(4) about 0.001-2% (w/w) preservative;
(5) about 65-90% (w/w) aqueous phase; and
(6) about 0.01-10% (w/w) cosmetic or pharmaceutical active ingredient.

In a thirty-ninth embodiment the invention provides a cosmetic or pharmaceutical topical formulation wherein said topical formulation comprises:
(1) about 10-20% (w/w) oil;
(2) about 3% (w/w) emulsifying agent;
(3) about 10% (w/w) permeation enhancer;
(4) about 0.01-1% (w/w) (e.g., about 0.01-1%, about 0.1-0.5%, or about 0.2%) preservative;
(5) about 82% (w/w) aqueous phase; and
(6) about 0.05-5%, (w/w) (e.g., about 0.05-5%, about 0.1-1%, or about 0.5-1%) cosmetic or pharmaceutical active ingredient.

In a fortieth embodiment the invention provides a cosmetic or pharmaceutical topical formulation according to the thirty ninth embodiment of the invention, wherein said topical formulation comprises:
(1) about 15% (w/w) oil;
(2) about 3% (w/w) emulsifying agent;
(3) about 10% (w/w) permeation enhancer;
(4) about 0.1-0.5%, (w/w) preservative;
(5) about 82% (w/w) aqueous phase; and
(6) about 0.1-1%, (w/w) cosmetic or pharmaceutical active ingredient.

In a forty first embodiment the invention provides a cosmetic or pharmaceutical topical formulation according to the thirty fifth embodiment of the invention, wherein said topical formulation comprises:
(1) about 15% (w/w) oil;
(2) about 3% (w/w) emulsifying agent;
(3) about 10% (w/w) permeation enhancer;
(4) about 0.01-1%, (w/w) preservative;
(5) about 82% (w/w) aqueous phase; and
(6) about 0.5-1% (w/w) cosmetic or pharmaceutical active ingredient.

In a forty second embodiment the invention provides a cosmetic or pharmaceutical topical formulation according to the thirty fifth embodiment of the invention, wherein said topical formulation comprises:
(1) about 15% (w/w) oil;
(2) about 3% (w/w) emulsifying agent;
(3) about 10% (w/w) permeation enhancer;
(4) about 0.1-0.5%, (w/w) preservative;
(5) about 82% (w/w) aqueous phase; and
(6) about 0.5-1% (w/w) cosmetic or pharmaceutical active ingredient.

In a forty third embodiment the invention provides a cosmetic or pharmaceutical topical formulation according to the thirty fifth embodiment of the invention, wherein said topical formulation comprises:
(1) about 15% (w/w) oil;
(2) about 3% (w/w) emulsifying agent;
(3) about 10% (w/w) permeation enhancer;
(4) about 0.2%, (w/w) preservative;
(5) about 82% (w/w) aqueous phase; and
(6) about 0.1-1% (w/w) cosmetic or pharmaceutical active ingredient.

In a forty fourth embodiment the invention provides a cosmetic or pharmaceutical topical formulation according to the thirty fifth embodiment of the invention, wherein said topical formulation comprises:
(1) about 15% (w/w) oil;
(2) about 3% (w/w) emulsifying agent;
(3) about 10% (w/w) permeation enhancer;
(4) about 0.2%, (w/w) preservative;
(5) about 82% (w/w) aqueous phase; and
(6) about 0.05-5% (w/w) cosmetic or pharmaceutical active ingredient.

In a forty fifth embodiment the invention provides a topical formulation according to any of the forgoing embodiments of the invention, wherein said topical formulation comprises:
(1) about 0.1-0.5%, (w/w) preservative; and
(2) about 0.1-1%, (w/w) cosmetic or pharmaceutical active ingredient.

In a forty sixth embodiment the invention provides a topical formulation according to any of the forgoing embodiments of the invention, wherein said topical formulation is an oil-in-water formulation.

In a forty seventh embodiment the invention provides a topical formulation according to any of the forgoing embodiments of the invention, wherein said topical formulation is a cream, a lotion, a gel, or a gel-cream.

In a forty eighth embodiment the invention provides a topical formulation according to any of the forgoing embodiments of the invention, wherein the mucosal surface is mucosa in the mouth, vaginal mucosal surface, mucosa of the eye.

In a forty ninth embodiment the invention provides a topical formulation according to any of the forgoing embodiments of the invention, wherein the active ingredient is: Compound 1, Compound 2; or wherein the active ingredient is effective to treat psoriasis, plaque psoriasis, nail psoriasis, psoriatic arthritis, hidradenitis suppurativa, alopecia areata/universalis, vitiligo, AKT keratosis (age spots), itch, or atopic dermatitis.

In a fiftieth embodiment the invention provides a topical formulation according to any of the forgoing embodiments of the invention, wherein the active ingredient is Compound 1,

In a fifty first embodiment the invention provides a topical formulation according to any of the forgoing embodiments of the invention, wherein the active ingredient is Compound 2,

In a fifty second embodiment the invention provides a cosmetic or pharmaceutical topical formulation prepared using the methods of the invention.

In a fifty third embodiment the invention provides a method for treating a skin or mucosal surface lesion, the method comprising applying to the lesion a topical formulation of the invention.

In a fifty fourth embodiment the invention provides a method according to the fiftieth embodiment of the invention, wherein the lesion is caused by psoriasis, plaque psoriasis, nail psoriasis, psoriatic arthritis, hidradenitis suppurativa, alopecia areata/universalis, vitiligo, AKT keratosis (age spots), itch, or atopic dermatitis.

In a fifty fifth embodiment the invention provides a use of a topical formulation according to any of the forgoing embodiments, in the manufacture of a medicament for treating a skin or mucosal surface lesion. In a specific embodiment, the lesion is caused by psoriasis, plaque psoriasis, nail psoriasis, psoriatic arthritis, hidradenitis suppurativa, alopecia areata/universalis, vitiligo, AKT keratosis (age spots), itch, or atopic dermatitis.

### BRIEF DESCRIPTION OF THE DRAWINGS

FIG. 1 shows solubility of a representative API (i.e., Compound 1) in the various permeation enhancers (PEs), water, and receptor media (PBS, pH 7.4, with 2% BSA). DMI: Dimethyl isosorbide; IPM: Isopropyl myristate; TC HP: TRANSCUTOL™ HP.

FIG. 2A shows 1% (w/w) Compound 1 transport across TUFFRYN® membrane in Franz Cells, in the presence of 10% different permeation enhancers in water. DMI: Dimethyl isosorbide; IPM: Isopropyl myristate; TC: TRANSCUTOL™.

FIG. 2B shows 0.1% (w/w) Compound 1 transport across TUFFRYN® membrane in Franz Cells, in the presence of 10% different permeation enhancers in water. DMI: Dimethyl isosorbide; IPM: Isopropyl myristate; TC: TRANSCUTOL™.

FIG. 3A shows 1% (w/w) Compound 1 transport across the various synthetic membranes, in the absence of permeation enhancer. Square data points:TRANSWELL® polycarbonate membrane; Triangle data points: TRANSWELL® polyester membrane; Diamond data points: Franz Cell TUFFRYN® membrane.

FIG. 3B shows 0.1% (w/w) Compound 1 transport across the various synthetic membranes, in the absence of permeation enhancer. Square data points: TRANSWELL® polycarbonate membrane; Triangle data points: TRANSWELL® polyester membrane; Diamond data points: Franz Cell TUFFRYN® membrane.

FIG. 4 shows Compound 1 flux through various synthetic membranes, in the absence of a permeation enhancer.

FIG. 5A shows 1% (w/w) Compound 1 transport across the various synthetic membranes, in the presence of a permeation enhancer—10% (w/w) TRANSCUTOL™ HP. Square data points represent TRANSWELL® polycarbonate membrane; Triangle data points represent TRANSWELL® polyester membrane; Diamond data points represent Franz Cell Tuffryn® membrane.

FIG. 5B shows 0.1% (w/w) Compound 1 transport across the various synthetic membranes, in the presence of a permeation enhancer—10% (w/w) TRANSCUTOL™ HP. Square data points represent TRANS WELL® polycarbonate membrane; Triangle data points represent TRANSWELL® polyester membrane; Diamond data points represent Franz Cell Tuffryn® membrane.

FIG. 5C shows Compound 1 flux through various synthetic membranes, in the presence of a permeation enhancer—10% (w/w) TRANSCUTOL™ HP.

FIG. 6A shows 1% (w/w) Compound 1 transport across the various synthetic membranes, in the presence of a permeation enhancer—10% (w/w) DMI in water. Square data points represent TRANSWELL® polycarbonate membrane; Triangle data points represent TRANSWELL® polyester membrane; Diamond data points represent Franz Cell TUFFRYN® membrane.

FIG. 6B shows 0.1% (w/w) Compound 1 transport across the various synthetic membranes, in the presence of a permeation enhancer—10% (w/w) DMI in water. Square data points represent TRANSWELL® polycarbonate membrane; Triangle data points represent TRANSWELL® polyester membrane; Diamond data points represent Franz Cell TUFFRYN® membrane.

FIG. 6C shows Compound 1 flux through various synthetic membranes, in the presence of a permeation enhancer—10% (w/w) DMI in water.

FIG. 7A shows 1% (w/w) Compound 1 transport across the various synthetic membranes, in the presence of a permeation enhancer—10% (w/w) IPM in water. Square data points represent TRANSWELL® polycarbonate membrane; Triangle data points represent TRANSWELL® polyester membrane; Diamond data points represent Franz Cell TUFFRYN® membrane.

FIG. 7B shows 0.1% (w/w) Compound 1 transport across the various synthetic membranes, in the presence of a permeation enhancer—10% (w/w) IPM. Square data points represent TRANS WELL® polycarbonate membrane; Triangle data points represent TRANSWELL® polyester membrane; Diamond data points represent Franz Cell TUFFRYN® membrane.

FIG. 7C shows Compound 1 flux through various synthetic membranes, in the presence of a permeation enhancer—10% (w/w) IPM in water.

FIG. 8 shows a schematic drawing of the skin structure, showing the different layers including epidermis, dermis, and hypodermis.

FIG. 9A shows plasma PK data following a 177.8 mg topical dose of Compound 1 to rats.

FIG. 9B shows plasma PK data following a 177.8 mg topical dose of Compound 2 to rats. Formulation B has (and Formulation A does not have) 10% (w/w) TRANSCUTOL™ HP as permeation enhancer.

FIG. 10 shows the effect of ear skin thickness reduction with Cyclosporin A (CsA 0.01%, 0.1% and 1%) topical formulations applied at 1 hour prior to FITC challenge.

FIG. 11A shows the pharmacokinetic (PK) data in the ear skin thickness model, including the systemic total blood CsA concentration and the local ear skin concentration.

FIG. 11B shows that the observed effect is dose dependent.

FIG. 12 shows the % effect of ear skin thickness reduction with CsA (1%) topical formulation applied at 1 hour after FITC challenge.

FIG. 13 shows % effect of ear skin thickness reduction with CsA (0.1% and 1%) topical formulations applied at 6 hours after FITC challenge.

FIG. 14 shows the % effect of ear skin thickness with BMS DPP (0.005%, 0.01%, 0.1% and 1%) topical formulations applied at 1 hour prior to FITC challenge.

FIG. 15 shows the pharmacokinetic (PK) data in the ear skin thickness model, including the local ear skin concentration of BMS DPP.

FIG. 16 shows the % effect of ear skin thickness reduction with BMS DPP (0.01%, 0.1% and 1%) topical formulations applied at 4 hours after FITC challenge.

### DETAILED DESCRIPTION OF THE INVENTION

### 1. Overview

Topical formulations, including creams, gels, and ointments, may be an optional treatment for patients suffering from various skin diseases or conditions, such as psoriasis, psoriatic arthritis, atopic dermatitis, alopecia, or vitiligo. To achieve desired therapeutic effects, it is necessary to develop an appropriate topical formulation that enables API penetration into the skin, resulting in locally high concentrations at the site of action. Several parameters may influence the release of an API from its formulation, including API crystallinity, particle size, solubility, and formulation composition. To ensure that maximum range of the active ingredient can be formulated into the topical formulation, it may be necessary to develop a method for formulating topical formulations that does not require a heating step, e.g., any heating step that subjects the active ingredients to a temperature higher than 35° C.

Thus one aspect of the invention provides a method for producing a formulation for topical application to skin or mucosal surface, the method comprising: (1) mixing an oil phase with an emulsifying agent to form a mixture, and agitating the mixture until homogeneous; (2) adding an aqueous phase and continuing to agitate to form a placebo formulation; (3) adding a solid form active ingredient (sAI) to the placebo formulation, and mixing until homogeneous; wherein the method does not comprise a step that subjects the sAI to a temperature higher than 35° C., e.g., the sAI is subjected to a temperature between about 15-30° C., or the sAI is subjected to a temperature about 25° C. In certain embodiments, the aqueous phase is purified water.

The emulsifying agent is a pharmaceutically acceptable surfactant, which may be a small molecule, oligomer or polymer. It may be nonionic, cationic or anionic. It may be of natural or synthetic origin.

Numerous emulsifying agents may be used in the instant invention. In certain embodiments, the emulsifying agent may comprise: sodium lauryl sulfate, or a non-ionic emulsifier (such as glyceryl stearate and/or PEG 100 stearate).

Other representative emulsifiers include, but are not limited to, gelatin, casein, lecithin (phosphatides), gum acacia, cholesterol, tragacanth, polyoxyethylene alkyl ethers, e.g., macrogol ethers such as cetomacrogol 1000, polyoxyethylene castor oil derivatives, polyoxyethylene sorbitan fatty acid esters, e.g., the commercially available Tweens, polyoxyethylene stearates, colloidal silicon dioxide, sodium dodecylsulfate, carboxymethylcellulose calcium, carboxymethylcellulose sodium, methylcellulose, hydroxyethylcellulose, hydroxypropylcellulose, microcrystalline cellulose, and magnesium aluminum silicate. Most of these surface modifiers are known pharmaceutical excipients and are described in detail in the Handbook of Pharmaceutical Excipients, published jointly by the American Pharmaceutical Association and The Pharmaceutical Society of Great Britain, the Pharmaceutical Press, 1986.

Other examples of surfactants include tyloxapol, poloxamers such as Pluronic F68, F77, and F108, which are block copolymers of ethylene oxide and propylene oxide, and polyxamines such as Tetronic 908 (also known as Poloxamine 908), which is a tetrafunctional block copolymer derived from sequential addition of propylene oxide and ethylene oxide to ethylenediamine, available from BASF, lecithin, dialkylesters of sodium sulfosuccinic acid, such as Aerosol OT, which is a dioctyl ester of sodium sulfosuccinic acid, available from American Cyanamid, Duponol P, which issodium lauryl sulfate, available from DuPont, Triton X-200, which is an alkyl aryl polyether sulfonate, available from Rohm and Haas, Tween® 20 and Tween® 80, which are polyoxyethylene sorbitan fatty acid esters, available from Croda, Inc.; Crodesta F-110, which is a mixture of sucrose stearate and sucrose distearate, available from Croda, Inc., Crodesta SL-40, which is available from Croda, Inc., and SA9OHCO, which is C₁₈H₃₇—CH₂(CON(CH₃)CH₂(CHOH)₄CH₂OH)₂, decanoyl-N-methylglucamide; n-decyl-β-D-glucsopyranoside; n-decyl-β-D-maltopyranoside; n-dodecyl-β-D-glucopyranoside; n-dodecyl-β-D-maltoside; heptanoyl-N-methylglucamide; n-heptyl-β-D-glucopyranoside; n-heptyl-β-D-thioglucoside; n-hexyl-β-D-glucopyranoside; nonanoyl-N-methylglucamide; n-noyl-β-D-glucopyranoside; octanoyl-N-methylglucamide; n-octyl-β-D-glucopyranoside; octyl-β-D-thioglucopyranoside; and the like.

In certain embodiments, the emulsifying agent may also be a thickener, a stabilizing agent, or both. Thus in one embodiment, the emulsifying agent may comprise a SEPINEO™ P 600-type hydro swelling droplet (HSD) polymer in an inverse emulsion (e.g., acrylamide/sodium acryloyldimethyl taurate copolymer/isohexadecane/polysorbate 80, or SEPINEO™ P 600 brand HSD polymer).

In other embodiments, the method may further comprise adding a thickener and/or a stabilizing agent in step (1) or step (2). This is, depending on the type of thickener/stabilizer selected, the thickener/stabilizer could be added to either the oil or aqueous phase.

In a specific embodiment, the thickener does not require heating, or at least does not require heating above 35° C. (e.g., a temperature between about 15-30° C., or a temperature about 25° C.), for use in a topical formulation.

In another specific embodiment, the thickener comprises one or more of: silicon dioxide (e.g., AEROSIL® brand silicon dioxide); xanthan gum; high molecular weight, crosslinked, acrylic acid-based polymers; polyvinyl alcohol; agarose; alginate; carrageenan; guar gum; cellulose derivatives; methylcellulose; sodium carboxymethylcellulose; hydroxypropyl cellulose; hydroxypropyl methylcellulose; hydroxyethyl cellulose; copovidone; and povidone.

In certain specific embodiments, the thickener comprises: high molecular weight, crosslinked, acrylic acid-based polymers; and/or cellulose derivatives, such as methylcellulose, sodium carboxymethylcellulose, hydroxypropyl cellulose, hydroxypropyl methylcellulose, and hydroxyethyl cellulose. Representative high molecular weight, crosslinked, acrylic acid-based polymers include carbomers or polyacrylates (e.g., CARBOPOL® brand polyacrylates).

As to stabilizing agents, in certain embodiments, the stabilizing agent may comprise: Plantacare 1200 UP (lauryl glucoside, e.g., BASF), Plantacare 2000 UP (Decyl Glucoside, e.g., BASF), Miranol Ultra 32 (Sodium cocoamphoacetate, e.g., Beiersdorf AG), Tego Care 450 (polyeglyceryl 3-methylglucose distearate, e.g., Goldschmidt AG), Tego Care CG 90 (cetearyl glucoside, e.g., Goldschmidt AG), Inutec SP1 (Inulin Lauryl Carbamate, e.g., Orafti), lecithin and its derivatives, purified phospholipids, Tween® 80 (polysorbate 80 or polyoxyethylene 20 sorbitan monooleate), or mixtures thereof.

In certain embodiments, the stabilizing agent may be cosmetic grade excipients. In other embodiments, the stabilizing agent may be pharmaceutical grade excipients.

In certain embodiments, the stabilizing agent comprises: Polyoxyethylene 20 sorbitan monooleate (e.g., that of TWEEN® 80 brand or equivalent); polyoxyethylene 20 sorbitan monolaurate (e.g., that of TWEEN® 20 brand or equivalent); polyoxyethylene 4 sorbitan monolaurate (e.g., that of TWEEN® 21 brand or equivalent); polyoxyethylene 20 sorbitan monopalmitate (e.g., that of TWEEN® 40 brand or equivalent); polyoxyethylene 5 sorbitan monooleate (e.g., that of TWEEN® 81 brand or equivalent); polyoxyethylene 20 sorbitan trioleate (e.g., that of TWEEN® 85 brand or equivalent); sorbitan monolaurate (e.g., that of SPAN® 20 brand or equivalent); sorbitan monooleate (e.g., that of SPAN® 80 brand or equivalent); sorbitan trioleate (e.g., that of SPAN® 85 brand or equivalent); polyoxyl 35 castor oil (e.g., that of KOLLIPHOR® EL brand or equivalent); and mixtures thereof.

In certain embodiments, the stabilizing agent comprises a polyoxyethylene sorbitan fatty acid ester.

In certain embodiments, the stabilizing agent comprises a polyoxyethylene castor oil derivative.

In certain embodiments, the stabilizing agent comprises a pharmaceutically acceptable liquid or solid stabilizer that can be processed at a temperature no higher than 35° C. or ambient conditions, e.g., a temperature between about 15-30° C., or a temperature about 25° C., for use in topical formulation.

In certain embodiments, the method further comprises, before step (2), adding a preservative to the aqueous phase. The numerous preservatives that can be used with the instant invention may comprise (but are not limited to): sodium benzoate, benzoic acid, sorbic acid, benzethonium chloride, benzalkonium chloride, bronopol, methylparaben, ethylparaben, propylparaben, butylparaben, thiomerosol, sodium propionate, chlorhexidine, chlorobutanol, chlorocresol, cresol, imidazolidinyl urea, diazolidinyl urea, phenol, phenylmercuric salts, potassium sorbate, propylene glycol, or mixtures thereof. In a specific embodiment, the preservative comprises imidazolidinyl urea.

In certain embodiments, the method further comprises adding a permeation enhancer to the mixture of step (1), between steps (1) and (2), to the aqueous phase of step (2), or between steps (2) and (3), partly depending on drug solubility, and/or the permeation enhancer selected. For example, the permeation enhancer may comprise: dimethyl isosorbide, isopropyl myristate, diethylene glycol monoethyl ether (e.g., TRANSCUTOL® HP brand diethylene glycol monoethyl ether), ethyl alcohol, ethyl oleate, isostearyl alcohol, oleyl alcohol, polyethylene glycol, N-methyl-2-pyrrolidone, dimethyl sulfoxide, or propylene carbonate.

In certain embodiments, the method further comprises measuring droplet particle size periodically during step (2). The agitation in step (2) may be partly dependent on the average droplet particle size and/or size distribution, and may be terminated or lengthened based on a pre-determined droplet particle size or distribution. The droplet particle size can be measured using any art-recognized method and instruments, such as optical microscopy. In certain embodiments, the droplet particle size may be measured once every minute, every 2, 3, 4, 5, 6, 7, 8, 9, 10, 12, 13, 14, 15, 16, 17, 18, 19, 20, 25, 30 minutes, etc.

In certain embodiments, the formulation is an oil-in-water emulsion, or a water-in-oil emulsion. For example, the SEPINEO™ P 600-based gel-creams usually result in oil-in-water systems. However, different emulsifiers/and emulsifier blends (stabilizers) may yield in either an oil-in-water or water-in-oil system.

In certain embodiments, the formulation is a lotion, a cream, a gel, or a gel-cream. In other embodiments, the formulation is in the form of ointments; emulsions, preferably in the form of creams, milks or pomades; powders, impregnated pads or adherent applicators such as patches, solutions, gels, sprays, lotions, suspensions, soaps, and shampoos. They may also be in the form of suspensions of microspheres or nanospheres or of lipid or polymer vesicles or of polymer patches and/or of hydrogels allowing controlled release. These compositions may be in anhydrous form, in aqueous form or in the form of an emulsion.

In certain embodiments, the oil phase is about 1-30% (w/w), about 10-20% (w/w), or about 15% (w/w) of the formulation.

In certain embodiments, the emulsifying agent is about 1-5% (w/w), or about 3% of the formulation.

In certain embodiments, when present, the preservative is about 0.001-2% (w/w), of the formulation.

In certain embodiments, when present, the preservative is about 0.01-1% (w/w of the formulation.

In certain embodiments, when present, the preservative is about 0.1-0.5% (w/w), of the formulation.

In certain embodiments, when present, the preservative is about 0.2% of the formulation.

In certain embodiments, when present, the permeation enhancer is less than 15% (w/w), of the formulation.

In certain embodiments, when present, the permeation enhancer is about 1-10% of the formulation.

In certain embodiments, the sAI is about 0.01-10% (w/w), about 0.05-5% (w/w), about 0.1-1% (w/w), or about 0.5-1% (w/w) of the formulation. In a specific embodiment, the sAI is about 0.5-1% (w/w) of the formulation.

In certain embodiments, the oil phase comprises a pharmaceutically acceptable oil that can be processed at a temperature no higher than 35° C. or ambient conditions, e.g., a temperature between about 15-30° C., or a temperature about 25° C., for use in a topical formulation. The oil phase may comprise: cod liver oil, light mineral oil, heavy mineral oil, shark liver oil, caprylic/capric triglyceride, vegetable oil, or mixtures thereof. The vegetable oil, for example, may comprise: castor oil, corn oil, canola oil, cottonseed oil, peanut oil, sesame oil, or soybean oil.

In certain embodiments, the oil phase comprises a biocompatible oil, such as triacetin, diacetin, tocopherol, or mineral oil. Other biocompatible oils that may be used include such oils listed in U.S. Pat. No. 5,633,226 (incorporated in its entirety by reference herein), and include CAPTEX™ 200, WHITEPSOL™ H-15 and MYVACET™ 9-45K, hydrogenated cocoa oil, coconut oil, elm seed oil, palm oil, cottonseed oil, soybean oil, parsley seed oil, mustard seed oil, linseed oil, tung oil, pomegranate seed oil, laurel oil, rapeseed oil, corn oil, evening primrose oil, maize oil, olive oil, persic oil, poppy-seed oil, safflower oil, sesame oil, soya oil, sunflower oil, ethyl oleate oil, Japanese anise oil, oil of eucalyptus, rose oil, almond oil, arachis oil, castor oil, mineral oil, peanut oil, vegetable oil and derivatives, sucrose polyester, silicone oil, and paraffin oil.

In certain embodiments, the sAI is a cosmetic or pharmaceutical active ingredient. Exemplary sAI may include compounds such as Compound 1, Compound 2, and any other small molecule inhibitors for the JAK family of kinases as disclosed in WO 2009/152133 and WO 2011/068881 (incorporated herein by reference), any of which can be used as API in the instant topical formulation, cyclosporine A or betamethasone dipropionate.

In certain embodiments, the sAI is effective to treat a skin or mucosal disease or condition, including but not limited to: psoriasis, plaque psoriasis, nail psoriasis, psoriatic arthritis, hidradenitis suppurativa, alopecia areata/universalis, vitiligo, AKT keratosis (age spots), itch, and atopic dermatitis.

In certain embodiments, the sAI is nitroglycerine (angina), scopolamine (motion sickness), fentanyl (pain control), nicotine (smoking cessation), estrogen (hormone replacement therapy), testosterone (male hypogonadism), clonidine (hypertension), and lidocaine (topical anesthesia).

In certain embodiments, the sAI is a topical acne agent (e.g., salicylic acid, tretinoin, benzoyl peroxide, erythromycin, benzoyl peroxide/sulfur, benzoyl peroxide/hydrocortisone, clindamycin, benzoyl peroxide/clindamycin, azelaic acid, benzoyl peroxide/erythromycin, adapalene, clindamycin/tretinoin, dapsone, benzoyl peroxide/salicylic acid, adapalene/benzoyl peroxide, clindamycin/tretinoin, resorcinol/sulfur, meclocycline sulfosalicylate, benzoyl peroxide/sodium hyaluronate, or combinations thereof), a topical anesthetic (e.g., benzalkonium chloride/lidocaine, lidocaine, lidocaine/prilocaine, benzocaine, pramoxine, tetracaine, lidocaine/prilocaine, dibucaine, hydrocortisone/lidocaine, phenol, calamine/pramoxine, benzalkonium chloride/lidocaine, pentafluoropropane/tetrafluoroethane, lidocaine/menthol, lidocaine/tetracaine, aloe vera/collagen/lidocaine, capsaicin/lidocaine/menthol, hydrocortisone/lidocaine/psyllium, or combinations thereof), a topical anti-infective (e.g., malathion, ivermectin, spinosad, silver, sinecatechins, docosanol, acetic acid, imiquimod, permethrin, piperonyl butoxide/pyrethrins, aloe polysaccharides/iodoquinol, chloroxine, crotamiton, nitrofurazone, cadexomer iodine, benzyl alcohol/zinc acetate, benzyl alcohol, or combinations thereof), a topical anti-rosacea agent (e.g., metronidazole, azelaic acid, brimonidine, or combinations thereof), a topical antibiotic (e.g., sulfacetamide sodium/sulfur, bacitracin/polymyxin b, erythromycin, sulfacetamide sodium, silver sulfadiazine, retapamulin, mupirocin, bacitracin/neomycin/polymyxin b, bacitracin/polymyxin b, neomycin/polymyxin b/pramoxine, bacitracin, sulfacetamide sodium/urea, neomycin/polymyxin b, sulfacetamide sodium/sulfur/urea, sulfacetamide sodium/urea, mafenide, tetracycline, or combinations thereof), a topical antifungal (e.g., tolnaftate, benzoic acid/salicylic acid, undecylenic acid, ketoconazole, naftifine, nystatin, miconazole, miconazole/zinc oxide, econazole, ciclopirox, oxiconazole, sertaconazole, efinaconazole, terbinafine, tavaborole, clotrimazole, sulconazole, salicylic acid/sodium thio sulfate, amphotericin b, chloroxylenol/undecylenic acid, haloprogin, clioquinol, luliconazole, butenafine, ketoconazole/pyrithione zinc, or combinations thereof), a topical anti-histamine (e.g., calamine/diphenhydramine, diphenhydramine, doxepin, or combinations thereof), a topical anti-neoplastic (e.g., fluorouracil, ingenol, imiquimod, mechlorethamine, or combinations thereof), a topical anti-psoriatic (e.g., tazarotene, betamethasone/calcipotriene, calcipotriene, calcitriol, anthralin, methoxsalen, resorcinol, or combinations thereof), a topical antiviral (e.g., acyclovir, penciclovir, or combinations thereof), a topical astringent (e.g., agents that cause skin cells or mucus membranes to contract or shrink, by precipitating proteins from their surface), a topical debriding agent (e.g., papain/urea, balsam peru/castor oil/trypsin, chlorophyllin copper complex/papain/urea, collagenase, or combinations thereof), a topical depigmenting agent (e.g., fluocinolone/hydroquinone/tretinoin, hydroquinone, monobenzone, or combinations thereof), a topical emollient (e.g., emollients, urea, ammonium lactate, salicylic acid/urea, vitamins a, d, and e, ammonium lactate/pramoxine, vitamin a & d, ammonium lactate/urea, salicylic acid/urea, aloe vera, lanolin, or combinations thereof), a topical keratolytic (e.g., salicylic acid, podofilox, podophyllum resin, trichloroacetic acid, or combinations thereof), a topical non-steroidal anti-inflammatory (e.g., diclofenac), a topical photochemotherapeutic (e.g., aminolevulinic acid, methyl aminolevulinate, methoxsalen, or combinations thereof), a topical rubefacient (e.g., methyl salicylate, trolamine salicylate, menthol, camphor/menthol, capsaicin/menthol/methyl salicylate, menthol/methyl salicylate, camphor, or combinations thereof), a topical steroid (e.g., triamcinolone, fluocinolone, desonide, betamethasone, halcinonide, hydrocortisone, diflorasone, clobetasol, desoximetasone, mometasone, clobetasol, fluticasone, fluocinonide, clocortolone, flurandrenolide, halobetasol, alclometasone, diflorasone, hydrocortisone/salicylic acid/sulfur, amcinonide, methylprednisolone, ammonium lactate/mometasone, ammonium lactate/halobetasol, or combinations thereof), a topical steroids with anti-infective (e.g., nystatin/triamcinolone, betamethasone/clotrimazole, clioquinol/hydrocortisone, aloe vera/hydrocortisone/iodoquinol, hydrocortisone/neomycin/polymyxin b, bacitracin/hydrocortisone/neomycin/polymyxin b, hydrocortisone/iodoquinol, acyclovir/hydrocortisone, fluocinolone/neomycin, hydrocortisone/ketoconazole, or combinations thereof), or a miscellaneous topical agent (e.g., menthol/zinc oxide, aluminum chloride hexahydrate, pyrithione zinc, zinc oxide, diphenhydramine/hydrocortisone, sodium hyaluronate, bimatoprost, salicylic acid/sulfur, coal tar, eflornithine, aluminum chloride hexahydrate, capsaicin, selenium sulfide, pimecrolimus, minoxidil, tacrolimus, bentoquatam, allantoin/camphor/phenol, coal tar/salicylic acid/sulfur, calamine, formaldehyde, salicylic acid/sulfur, sulfur, lactic acid, alitretinoin, dexpanthenol, becaplermin, mequinol/tretinoin, bexarotene, balsam peru/castor oil, coal tar/salicylic acid, or combinations thereof).

In certain embodiments, the permeation enhancer comprises: dimethyl isosorbide, isopropyl myristate, diethylene glycol monoethyl ether (e.g., TRANSCUTOL® HP brand diethylene glycol monoethyl ether), ethyl alcohol, ethyl oleate, isostearyl alcohol, oleyl alcohol, polyethylene glycol, N-methyl-2-pyrrolidone, dimethyl sulfoxide, or propylene carbonate.

In certain embodiments, the method further comprises, after Step (1) or Step (2), adding an sAI and mixing until homogenous.

In certain embodiments, the method further comprising adding one or more inert additives or combinations thereof, including but not limited to: wetting agents; texture enhancers; humidity regulators; pH regulators; osmotic pressure modifiers; UV-A and UV-B screening agents; and antioxidants. For example, antioxidants can be α-tocopherol, butylated hydroxyanisole or butylated hydroxytoluene, superoxide dismutase, ubiquinol, or certain metal-chelating agents. One skilled in this art will be able to select the optional compound(s) to be added to these compositions such that the advantageous properties intrinsically associated with the present invention are not, or are not substantially, adversely affected by the envisaged addition.

In a specific embodiment, the method comprises: (1) mixing an oil phase comprising about 15% (w/w) light mineral oil, with an emulsifying agent comprising about 3% (w/w) SEPINIO P 600, HSD polymer to form a mixture in a first reactor, and agitating the mixture until homogeneous; (2) adding an aqueous phase and continuing to agitate to form a placebo formulation, wherein the aqueous phase is prepared by mixing in a second reactor about 0.2% (w/w) preservative comprising imidazolidinyl urea, about 10% (w/w) permeation enhancer comprising TRANSCUTOL® HP, and about 71.8% (w/w) purified water; and (3) adding a solid form active ingredient (sAI), e.g., about 1% (w/w), to the placebo formulation, and mixing until homogeneous; wherein the method does not comprise a step that subjects the sAI to a temperature higher than 35° C., e.g., the entire operation is conducted at around room temperature.

In certain embodiments, the mixing in step (2) of the aqueous phase with the homogeneous mixture of step (1) is conducted at 250 rpm in a 10 L reactor. Optionally, the resulting oil-in-water emulsion is periodically sampled, e.g., once every 12 minutes or so, to check droplet particle size by optical microscopy.

A related aspect of the invention provides a method for producing a placebo formulation that can be used for further preparation of the subject topical formulation for topical application to skin or mucosal surface, the method comprising: (1) mixing an oil phase with an emulsifying agent to form a mixture, and agitating the mixture until homogeneous; and (2) adding an aqueous phase and continuing to agitate to form the placebo formulation. The placebo formulation can be mixed with a solid form active ingredient to form the subject topical formulation, without exposing the solid form active ingredient to a temperature higher than 35° C.

Another aspect of the invention provides a cosmetic or pharmaceutical topical formulation for applying to skin or a mucosal surface, wherein the topical formulation comprises: (1) about 1-30% (w/w) (e.g., about 10-20%, or about 15%) oil (e.g., mineral oil); (2) about 1-5% (w/w) (e.g., 3%) emulsifying agent (e.g., a SEPINEO™ P 600-type hydro swelling droplet (HSD) polymer in an inverse emulsion (such as acrylamide/sodium acryloyldimethyl taurate copolymer/isohexadecane/polysorbate 80, or SEPINEO™ P 600 brand HSD polymer)); (3) about 1-15% (w/w) (e.g., 10%) permeation enhancer (e.g., diethylene glycol monoethyl ether or TRANSCUTOL® HP); (4) about 0.001-2% (w/w) (e.g., about 0.01-1%, about 0.1-0.5%, or about 0.2%) preservative (e.g., imidazolidinyl urea); (5) about 65-90% (w/w) (e.g., 82%) aqueous phase (e.g., purified water); (6) about 0.01-10% (w/w) (e.g., about 0.05-5%, about 0.1-1%, or about 0.5-1%) cosmetic or pharmaceutical active ingredient.

In certain embodiments, the topical formulation is an oil-in-water formulation.

In certain embodiments, the topical formulation is a cream, a lotion, a gel, or a gel-cream.

In certain embodiments, the mucosal surface is mucosa in the mouth, vaginal mucosal surface, mucosa of the eye or mucosa of the nose.

In certain embodiments, the topical formulation comprises: (1) about 15% (w/w) oil (e.g., mineral oil); (2) about 3% (w/w) emulsifying agent (e.g., SEPINEO™ P 600); (3) about 10% (w/w) permeation enhancer (e.g., diethylene glycol monoethyl ether or TRANSCUTOL® P (>99.7% purity) or HP (>99.9% purity)); (4) about 0.2% (w/w) preservative (e.g., imidazolidinyl urea); (5) about 82% (w/w) purified water; and (6) about 0.5-1% (w/w) cosmetic or pharmaceutical active ingredient. Specific embodiments also include those disclosed in the examples or can be derived based on the examples (e.g., by adding one or more APIs to the placebo formulations in the examples), such as Examples 1-4.

In certain embodiments, the active ingredient of the topical formulation is: Compound 1, Compound 2, and any other small molecule inhibitors for the JAK family kinases as disclosed in WO 2009/152133 and WO 2011/068881, cyclosporine A or betamethasone dipropionate, any of which can be used as API in the instant topical formulation or wherein the active ingredient is effective to treat a skin or mucosal disease or condition, such as (without limitation): psoriasis, plaque psoriasis, nail psoriasis, psoriatic arthritis, hidradenitis suppurativa, alopecia areata/universalis, vitiligo, AKT keratosis (age spots), itch, or atopic dermatitis.

In certain embodiments, the cosmetic or pharmaceutical topical formulation further comprises one or more inert additives or combinations thereof, including but not limited to: wetting agents; texture enhancers; humidity regulators; pH regulators; osmotic pressure modifiers; UV-A and UV-B screening agents; and antioxidants. For example, the antioxidants can be α-tocopherol, butylated hydroxyanisole or butylated hydroxytoluene, superoxide dismutase, ubiquinol, or certain metal-chelating agents.

In a related aspect, the invention provides a cosmetic or pharmaceutical topical formulation prepared using any of the methods of the invention as described herein.

In another related aspect, the invention provides a placebo formulation useful for making the subject cosmetic or pharmaceutical topical formulation according to the methods of the invention, wherein the placebo formulation is substantially identical to the subject cosmetic or pharmaceutical topical formulation, except for lacking a pharmaceutically or cosmetically active ingredient. The various embodiments of the cosmetic or pharmaceutical topical formulations described herein above and below, sans the active ingredient, are incorporated herein.

Another aspect of the invention provides a method for treating a skin or mucosal surface lesion associated with a disease or condition, the method comprising applying to the lesion of any one of the topical formulations of the invention.

In certain embodiments, the lesion is caused by or is associated with psoriasis, plaque psoriasis, nail psoriasis, psoriatic arthritis, hidradenitis suppurativa, alopecia areata/universalis, vitiligo, AKT keratosis (age spots), itch, or atopic dermatitis.

With the invention being generally described above, specific aspects and embodiments of the invention are described in further detail below, with the understanding that any specific aspects and embodiments of the invention can be combined with any other specific aspects and embodiments of the invention, unless otherwise prohibited or disclaimed.

### 2. HSD Polymer

The SEPINEO™ P 600 brand HSD polymer is suitable for the subject topical drug formulation, in that it is a multi-functional agent that is simultaneously an emulsifier, a thickener, and a stabilizing agent. According to the manufacturer, it is a hydro swelling droplet (HSD) polymer in an inverse emulsion, wherein a discontinuous pre-neutralized polymer phase is evenly distributed within a continuous external oily phase. Inverted surfactants reside at the aqueous-oily interface and extending into the oily phase. In the presence of water, SEPINEO™ P 600-type HSD polymer reverses, and the polymer network deploys instantly, forming a stable gel in a few seconds. Thus it is able to emulsify and stabilize many oily phases without the addition of a conventional emulsifier, regardless of the nature of the oily phases (e.g., polar and non polar oils, vegetable oils, silicone oils, esters).

In certain embodiments, the gel-creams obtained are stable and have a uniform appearance, very pleasant to the touch and to spread on the skin.

In addition to its thickening/emulsifying properties, SEPINEO™ P 600 brand HSD polymer also stabilizes conventional emulsions. In certain embodiments, when the SEPINEO™ P 600 brand HSD polymer is used as a stabilizer, its concentration can be from about 0.5% to 2.0% (w/w), depending on the nature of the other ingredients of the formula.

For emulsion formulations, the SEPINEO™ P 600-type HSD polymer can be added either at the end of the emulsification step, or added at the beginning of the process either in the oil or aqueous phase.

For gel-cream formulations, the SEPINEO™ P 600-type HSD polymer can be introduced either in the oily phase or in the aqueous phase. However, the stirring step tends to need less energy when it is introduced in the oily phase.

For hydroglycolic/hydroalcoholic/acetone gels, it is advisable to prepare the aqueous gel first and then add the solvent gradually, while stirring.

The amount of the SEPINEO™ P 600-type HSD polymer can be adjusted depending on the type of formulation desired. For example, if a gel is desired, up to 5% (w/w) may be used to provide the necessary thickening power. Similarly, if a gel-cream is desired, up to 5% (w/w) may be used to provide the necessary emulsifying power. In cream formulations, about 0.5-2% (w/w) may be used to provide the desired stabilizing power.

### 3. Penetration Enhancers (PEs)

The greatest hindrance in the percutaneous delivery of API is the obstructive property of the stratum corneum (SC), the outermost layer of the skin, in addition to usual problems such as skin binding, skin metabolism, cutaneous toxicity and prolonged lag times.

Different methodologies have been developed to enhance transdermal absorption, including the use of drug derivatives, super-saturated systems, physical approaches, and chemical penetration enhancers (sorption promoters) that facilitate the diffusion of drugs through the SC. In that regard, numerous chemicals have been used for their skin permeation promoting capacity, including fatty acids, fatty acid esters, fatty alcohols or fatty alcohol ethers, fatty ethers, lower alcohols, glycerol esters, polyhydric alcohols, diols, amides (e.g., N,N-diethyl-m-toluamide), amines, terpenes, polar solvents, pyrrolidones and derivatives thereof, sulfoxides, azone or laurocapram, surface active agents, lecithin, polyols, glycols, quaternary ammonium compounds, silicones, alkanoates, certain biologics, enzymes, complexing agents, macrocyclics, solvents, etc., all of which can potentially be used in the topical formulations and methods of the invention. In certain embodiments, the permeation enhancers useful for the instant invention does not require heating, e.g., heating above 35° C., for use in the subject topical formulation and method of preparation thereof.

As used herein, “permeation enhancement” refers to increasing the permeability of the skin to a cosmetically or pharmacologically effective ingredient (or API), so as to increase the rate at which the API permeates through the skin. Similarly, “permeation enhancer” or simply PE refers to an agent or mixture of agents that achieve such permeation enhancement.

In certain embodiments, a PE suitable for the instant invention promotes penetration of an API through the skin by one or more of the following mechanisms: (1) by increasing the diffusivity of the drug in the skin; (2) by causing SC lipid-fluidization, which leads to decreased barrier function (a reversible action); (3) by increasing and optimizing the thermodynamic activity of the drug in the vehicle; (4) by affecting the partition coefficient of the drug; and (5) by increasing its release from the formulation into the upper layers of the skin.

In certain embodiments, a PE suitable for the instant invention has one or more of the following characteristics: non-toxic, non-irritant, non-allergenic, and/or non-sensitizing to skin; pharmacologically inert, at least at the concentrations required to exert adequate permeation action; immediate, predictive, and/or reversible effect; easily incorporated into pharmaceutical preparations; and cosmetically acceptable.

In certain embodiments, the PE is used in conjunction with an API at a subsaturated API concentration. In certain embodiments, the PE is used in conjunction with an API at its saturated or supersaturated concentrations.

In certain embodiments, the PE is a fatty acid, such as a long chain fatty acid, to promote delivery of both lipophilic and hydrophilic APIs. For example, the fatty acid may be oleic acid (cis-9-octadecenoic acid), or a functional derivative thereof. In certain embodiments, the PE is a fatty acid ester, fatty alcohol or fatty alcohol ether, fatty ether, lower alcohol, glycerol ester, polyhydric alcohol, diol, amide (e.g., N,N-diethyl-m-toluamide), amine, terpene, polar solvent or a mixture thereof.

In certain embodiments, the fatty acid is alkanoic acid, capric acid, diacid, ethyloctadecanoic acid, hexanoic acid, lactic acid, lauric acid, linoelaidic acid, linoleic acid, linolenic acid, neodecanoic acid, oleic acid (cis-9-octadecenoic acid), palmitic acid, pelargonic acid, propionic acid, or vaccenic acid. In certain embodiments, the PE is at least one of a C8-C22 fatty acid, such as isopropyl myristate.

In certain embodiments, the fatty alcohol ether is α-Monoglyceryl ether, EO-2-oleyl ether, EO-5-oleyl ether, EO-10-oleyl ether, or ether derivatives of polyglycerols and alcohols (e.g., 1-O-dodecyl-3-O-methyl-2-O-(2′,3′-dihydroxypropyl)glycerol).

In certain embodiments, the fatty acid ester is glycerol monolaurate, glycolic acid, lauroyl glycolic acid, butyl acetate, cetyl lactate, decyl N,N-dimethylamino acetate, decyl N,N-dimethylamino isopropionate, diethyleneglycol oleate, diethyl sebacate, diethyl succinate, diisopropyl sebacate, dodecyl N,N-dimethylamino acetate, dodecyl (N,N-dimethylamino)-butyrate, dodecyl N,N-dimethylamino isopropionate, dodecyl 2-(dimethylamino)propionate, EO-5-oleyl ester, ethyl acetate, ethyl acetoacetate, ethyl propionate, glycerol monoethers, glycerol monolaurate, glycerol monooleate, glycerol monolinoleate, isopropyl isostearate, isopropyllinoleate, isopropyl myristate, isopropyl myristate/fatty acid monoglyceride combination, isopropyl myristate/ethanol/L-lactic acid (87:10:3) combination, isopropyl palmitate, methyl acetate, methyl caproate, methyl laurate, methyl propionate, methyl valerate, 1-monocaproyl glycerol, monoglycerides (medium chain length), nicotinic esters (benzyl), octyl acetate, octyl N,N-dimethylamino acetate, oleyl oleate, n-pentyl N-acetylprolinate, propylene glycol monolaurate, sorbitan dilaurate, sorbitan dioleate, sorbitan monolaurate, sorbitan monooleates, sorbitan trilaurate, sorbitan trioleate, sucrose coconut fatty ester mixtures, sucrose monolaurate, sucrose monooleate, or tetradecyl N,N-dimethylamino acetate.

While not wishing to be bound by any particular theory, the fatty acid PEs of the invention are believed to selectively perturb the intercellular lipid bilayers in the SC, thus enhancing the penetration of the SC by the API.

In certain embodiments, differences in penetration enhancing effects may be adjusted by adjusting the number of double bonds and cis/trans configuration of the fatty acid isomers, based on the general trend that unsaturated fatty acids are more effective (e.g., more than 5-fold, 10-fold, 15-fold, 20-fold or more) in enhancing percutaneous absorption than their saturated counterparts, especially for lipophilic drugs/APIs.

In certain embodiments, the PE is oleic acid, linoleic acid, α-linolenic acid, arachidonic acid, palmitic acid, lauric acid, caprylic acid, isostearic acid, isopropyl myristate, or myristic acid, optionally further comprising one or more of propylene glycol, ethanol, 2-ethyl-1,3-hexanediol, and dexpanthene. In certain embodiments, the PE is palmitic acid, and the topical formulation is formulated to enhance the penetration of an API to the SC (a particularly alkyl-rich region). In certain embodiments, the PE is myristic acid, and the topical formulation is formulated to enhance the penetration of an API to the epidermis. In certain embodiments, the PE is octyl salicylate, and the topical formulation is formulated to enhance the penetration of a water-soluble or oil-soluble API into the epidermis and dermis.

In certain embodiments, the fatty acid PEs are substantially free of skin irritation, such as straight-chain saturated fatty acids.

Additional fatty acid based PEs can be found in MX 9705070, GR 1004995, US 2005-020552A1, WO 05/060540, CA 2,420,895, MX 9800545, WO 04/054552, NZ 537359, WO 98/18417, WO 96/30020, DE 4301783, U.S. Pat. No. 4,885,174, U.S. Pat. No. 4,983,396, NZ 222346, CA 1,280,974, and U.S. Pat. No. 4,626,539.

In certain embodiments, the PE is a terpene, that can be used to increase the percutaneous permeation parameters for a large number of lipophilic and hydrophilic compounds. While not wishing to be bound by any particular theory, the terpenes are believed to affect the intercellular packing of the SC lipids so as to change the barrier properties of the skin.

In certain embodiments, the terpene is linalool, cineole (e.g., eucalyptol), limonene (e.g., R—(C)-limonene, d-limonene), 1-menthol, forskolin, menthone, menthol, terpineol, geraniol, nerolidol, thymol, pyrrolidone ring derivative, alcoholic terpene (e.g., basil oil, methyl chavicol, eugenol, linalool, camphor, methyl cinnamate), clove oil, essential oil or a volatile oil extract from the genus Curcuma of the family Zingiberaceae, germacrone, or a natural or synthetic constituent thereof.

In certain embodiments, a secondary permeation enhancer of a polyalcohol, a monoalkyl ether of diethylene glycol, a tetraglycol or a mixture thereof is used for optimal permeation enhancement, for example, while terpene is the primary PE.

Additional terpene based PEs can be found in WO 08/058220, WO 05/105059, US 2005-244522, WO 90/08553, and U.S. Pat. No. 6,723,337.

In certain embodiments, the PE is a fatty alcohol or aliphatic alcohol, such as octanol, myristyl alcohol, decanol or decyl alcohol, undecanol or undecyl alcohol, tridecanol, n-octanol, i-nonanol, lauryl alcohol (dodecanol), oleyl alcohol, dodecyl alcohol propylene glycol, nerolidol, linolenyl alcohol, polyethylene glycol, C9-C11, C12-C13 or C12-C15 fatty alcohols, or mixtures thereof. In certain embodiments, the PE effect is adjusted based on the observation of a general increase in PE effect by adding up to two unsaturated bonds into the alcohols, and a declined activity when three double bonds are introduced. In certain embodiments, the PE is a non-polar functional derivative of a fatty acid, such as oleyl alcohol.

Additional fatty alcohol based PEs can be found in US 2007-212410, WO 07/100757, EP 0224981, and US 2007-065494.

In certain embodiments, the PE is a pyrrolidone or a derivative thereof, such as N-methyl-2-pyrrolidone (NMP) and 2-pyrrolidone (2P), for enhancing permeability of numerous hydrophilic and lipophilic APIs.

In certain embodiments, the PE is N-Cyclohexyl-2-pyrrolidone, 1-Butyl-3-dodecyl-2-pyrrolidone, 1,3-Dimethyl-2-imidazolikinone, 1,5-Dimethyl-2-pyrrolidone, 4,4-Dimethyl-2-undecyl-2-oxazoline, 1-Ethyl-2-pyrrolidone, 1-Hexyl-4-methyloxycarbonyl-2-pyrrolidone, 1-Hexyl-2-pyrrolidone, 1-(2-Hydroxyethyl)pyrrolidinone, 3-Hydroxy-N-methyl-2-pyrrolidinone, 1-isopropyl-2-undecyl-2-imidazoline, 1-Lauryl-4-methyloxycarbonyl-2-pyrrolidone, N-Methyl-2-pyrrolidone, poly(N-vinylpyrrolidone), pyroglutamic acid esters, or 2-Pyrrolidone (2-pyrrolidinone).

In certain embodiments, the pyrrolidone based PE is used alone or in combination with at least one skin penetration enhancer selected from the group consisting of oleic acid, oleyl alcohol, linoleic acid, isopropyl linoleate, azone, butanediol, and partially methylated β cyclodextrin (PMβCD).

Additional pyrrolidone based PEs can be found in U.S. Pat. No. 5,262,165 and EP 0417496.

In certain embodiments, the PE is a sulfoxide, such as dimethylsulfoxide (DMSO), for enhancing the penetration of both hydrophilic and lipophilic APIs.

While not wishing to be bound by any particular theory, DMSO is believed to enhance the permeation of active substances by one of the following means: by promotion of drug partitioning from the dosage form, it denatures the intercellular structural proteins of the SC, or promotes lipid fluidity by disruption of the ordered structure of the lipid chains, and may alter the physical structure of the skin by elution of lipid, lipoprotein and nucleoprotein structures of the SC. The activity of DMSO is concentration-dependent, and a positive response is observed with enhancer concentrations >60%.

Additional DMSO like PEs include similar, chemically related compounds such as Dimethylacetamide (DMAC), dimethylformamide (DMF), cyclic sulfoxides, decylmethyl sulfoxide, Dimethyl sulfoxide, and 2-Hydroxyundecyl methyl sulfoxide.

In certain embodiments, DMSO like PEs may be used with decylmethyl sulfoxide, N-dodecyl pyrrolidone, decanol, dodecanol or an organic acid. See WO 05/120407, WO 93/18752, and U.S. Pat. No. 6,113,921 for DMSO and related reagents as PEs.

In certain embodiments, the PE is an azone (1-dodecylazacycloheptan-2-one or laurocapram), or a derivative such as 1-n-dodecyl-azacycloheptan-2-thione. Azone is considered to be a hybrid of two potent permeation enhancers—pyrrolidone and decylmethylsulfoxide, and is known to show significant accelerant effects at low concentrations for both hydrophilic and hydrophobic APIs. While not wishing to be bound by any particular theory, azone is believed to exert its penetration enhancing effects through interactions with the lipid domains of the SC.

In certain embodiments, the azone is nonirritating and nonallergenic. In certain embodiments, the azone (e.g., about 2%) is in propylene glycol. In certain embodiments, azone is used in conjunction with another PE, such as a fatty acid (e.g., oleic acid), fatty acid ester, sunscreen ester, long chain alkyl (N,N-disubstituted amino) carboxylate, 1,3-dioxacyclopentane, or 1,3-dioxacyclohexane.

In certain embodiments, the azone is N-Acyl-hexahydro-2-oxo-1H-azepines, N-Alkyl-dihydro-1,4-oxazepine-5,7-diones, N-Alkylmorpholine-2,3-diones, N-Alkylmorpholine-3,5-diones, Azacycloalkane derivatives (-ketone, -thione), Azacycloalkenone derivatives, 1-[2-(Decylthio)ethyl]azacyclopentan-2-one (HPE-101), N-(2,2-Dihydroxyethyl)dodecylamine, 1-Dodecanoylhexahydro-1-H-azepine, 1-Dodecyl azacycloheptan-2-one (azone or laurocapram), N-Dodecyl diethanolamine, N-Dodecyl-hexahydro-2-thio-1-H-azepine, N-Dodecyl-N-(2-methoxyethyl)acetamide, N-Dodecyl-N-(2-methoxyethyl)isobutyramide, N-Dodecyl-piperidine-2-thione, N-Dodecyl-2-piperidinone, N-Dodecyl pyrrolidine-3,5-dione, N-Dodecyl pyrrolidine-2-thione, N-Dodecyl-2-pyrrolidone, 1-Farnesylazacycloheptan-2-one, 1-Farnesylazacyclopentan-2-one, 1-Geranylazacycloheptan-2-one, 1-Geranylazacyclopentan-2-one, Hexahydro-2-oxo-azepine-1-acetic acid esters, N-(2-Hydroxyethyl)-2-pyrrolidone, 1-Laurylazacycloheptane, 2-(1-Nonyl)-1,3-dioxolane, 1-N-Octylazacyclopentan-2-one, N-(1-Oxododecyl)-hexahydro-1H-azepine, N-(1-Oxododecyl)-morpholines, 1-Oxohydrocarbyl-substituted azacyclohexanes, N-(1-Oxotetradecyl)-hexahydro-2-oxo-1H-azepine, or N-(1-Thiododecyl)-morpholines.

See, for example, NZ 222346, U.S. Pat. No. 5,391,548, U.S. Pat. No. 4,886,783, MX PA06006041, U.S. Pat. No. 4,562,075, EP 0095169, U.S. Pat. No. 4,405,616, and U.S. Pat. No. 5,270,346, for use of azone or derivatives thereof as PE.

In certain embodiments, the PE is a surface active agent or surfactant, which functions primarily by adsorption at interfaces and thus interacts with biological membranes contributing to the overall penetration enhancement of compounds. In certain embodiments, the surfactant is a cationic surfactant or anionic surfactant. In certain embodiments, the surfactant is a nonionic surfactant.

Examples of surfactants include: sodium lauryl sulfate (sodium dodecyl sulfate), lauryl glucoside, polysorbate 20, cocoamidopropyl betaine, sorbitan mono-9-octadecenoate poly (oxy-1,2-ethanediyl) and its derivatives, a combination of sodium lauryl ether sulfate and 1-phenyl piperazine, a combination of N-lauryl sarcosine and Span 20/sorbitan monolaurate, ascorbate, amphoteric cations and anions, calcium thioglycolate, cetyl trimethyl ammonium bromide, 3,5-Diiodosalicylate sodium, ionic surfactants (ROONa, ROSO₃Na, RNH₃Cl), Lauroylcholine iodide, 5-Methoxysalicylate sodium, monoalkyl phosphates, 2-PAM chloride, 4-PAM chloride (derivatives of N.methyl picolinium chloride), sodium carboxylate, sodium hyaluronate, Span® 20, Tween® 20, and Tween® 80. See, for example, US 2007-269379 and JP 2003607 (all incorporated by reference).

In certain embodiments, the PE is a classical surfactant selected from the group consisting of: Brij 30, Brij 36T, Brij 35, Brij 52, Brij 56, Brij 58, Brij 72, Brij 76, Brij 78, Brij 92, Brij 96, Brij 98, cetyl trimethyl ammonium bromide, empicol ML26/F, HCO-60 surfactant, hydroxypolyethoxydodecane, ionic surfactant (ROONa, ROSO₃Na, RNH₃Cl, R=8-16), lauroyl sarcosine, nonionic surface active agent, nonoxynol, octoxynol, phenylsulfonate CA, Pluronic F68, Pluronic F127, Pluronic L62, polyoleates (nonionic surfactant), Rewopal® HV10, sodium laurate, sodium lauryl sulfate (sodium dodecyl sulfate), sodium oleate, sorbitan dilaurate, sorbitan dioleate, sorbitan monolaurate, sorbitan monooleate, sorbitan trilaurate, sorbitan trioleate, Span® 20, Span® 40, Span® 85, Synperonic® NP, Triton® X-100, Tween® 20, Tween® 40, Tween® 60, Tween® 80, and Tween® 85.

In certain embodiments, the PE is an amide or an amine. In certain embodiments, the PE includes one or more of: urea or a derivative thereof (e.g., unsaturated cyclic urea), ethanolamine, diethanolamine, triethanolamine, propanolamine, diisopropanolamine, triisopropanolamine, butanolamine, dibutanolamine, or tributanolamine; or an amide selected from the group consisting of coconut fatty acid diethanolamide and lauric fatty acid diethanolamide.

In certain embodiments, the PE is an acetamide derivative, acyclic amide, N-adamantyl n-alkanamide, clofibric acid amide, N,N-didodecyl acetamide, di-2-ethylhexylamine, diethyl methyl benzamide, N,N-diethyl-m-toluamide, N,N-dimethyl-m-toluamide, ethomeen S12 [bis-(2-hydroxyethyl)oleylamine], hexamethylene lauramide, lauryl-amine (dodocylamine), octyl amide, or oleylamine.

See, for example, US 2005-042268 and WO 04/026313 (all incorporated by reference).

In certain embodiments, the PE is lecithin. See, for example, WO 01/35927, U.S. Pat. No. 6,011,022, TW 265027B, and U.S. Pat. No. 6,143,278 (all incorporated by reference).

In certain embodiments, the PE is a polyol or glycol, such as propylene glycol, hexylene glycol, Sefsol® 318 (a medium-chain glyceride), alkylene glycol, propylene glycol, lauric diethanolamide, or polyethylene glycol. In certain embodiments, the PE is a conjugate of a fatty acid (such as an unsaturated fatty acid, e.g., oleic acid and linoleic acid) and propylene glycol, such as oleate-propylene glycol and linoeate-propylene glycol mono-conjugates.

In a specific embodiment, the PE is a diethylene glycol monoethyl ether, such as the one marketed as TRANSCUTOL® brand diethylene glycol monoethyl ether by GATTEFOSSE SAS (France) with an optional second PE such as propylene glycol, myristyl alcohol, unsaturated polyglycolysed glyceride, glyceryl and polyethylene glycol ester, propylene glycol laurate, oil of Melaleuca, propylene glycol, 2-methyl-1,3-propanediol polyethylene glycol, or a combination thereof. See, for example, WO 96/19976, MX 9707868, U.S. Pat. No. 5,916,587, WO 08/012071, WO 08/005240, and US 2005-287195.

In certain embodiments, the PE is a quaternary ammonium compound, such as benzalkonium chloride, stearalkonium, behenalkonium chloride, olealkonium chloride, erucalkonium chloride, benzethonium chloride, methylbenzethonium chloride, phenoctide or a mixture thereof. In certain embodiments, the PE is used with one or more co-enhancers selected from the group consisting of: fatty acids and their salts, fatty alcohols, branched aliphatic alcohols, fatty acid alkyl esters, fatty acid monoesters of sorbitol and glycerol, fatty acid esters with glycolic acid and lactylic acid and their salts, fatty acid amides, alkylpyrrolidones and mixtures thereof, which may provide a synergistic skin permeation enhancing effect when combined with the quaternary ammonium salt. See, for example, US 2005-025833 and US 2003-091620.

In certain embodiments, the PE is a silicone. In certain embodiments, the PE is selected from the group consisting of: a dimethicone, a cyclomethicone, a simethicone, and an oligodimethylsiloxane. See, for example, CA2602018.

In certain embodiments, the PE is an alkanoate. In certain embodiments, the PE is an alkyl-2-(N,N-disubstituted amino)-alkanoate ester such as dodecyl (N,N-dimethylamino)-acetate, or an (N, N-disubstituted amino)-alkanol alkanoate such as dodecyl 2-(N,N-dimethylamino)-propionate, or a mixture thereof. See, for example, MX PA01011560 and CA 2,442,479.

In certain embodiments, the PE is a biologic origin, such as L-α-amino-acid, lecithin, phospholipid, Saponin/phospholipid, sodium deoxycholate, sodium taurocholate, or sodium tauroglycocholate.

In certain embodiments, the PE is an enzyme, such as acid phosphatase, calonase, orgelase, papain, phospholipase A-2, phospholipase C, or triacylglycerol hydrolase.

In certain embodiments, the PE is a complexing agent, such as β- or γ-cyclodextrin and their derivatives, hydroxypropyl methylcellulose liposome, naphthalene diamide diimide, or naphthalene diester diimide.

In certain embodiments, the PE is a macrocyclic, such as macrocyclic lactone, ketone, anhydride (optimum ring-16), or unsaturated cyclic ureas.

In certain embodiments, the PE is a solvent or a related compound selected from the group consisting of: acetamide and derivatives, acetone, n-alkane (e.g., chain length between 7 and 16), alkanol, diol, short-chain fatty acid (e.g., fewer than 6 carbons), cyclohexyl-1,1-dimethylethanol, dimethyl acetamide, dimethyl formamide, ethanol, ethanol/d-limonene combination, 2-ethyl-1,3-hexaediol, ethoxydiglycol (TRANSCUTOL® P or HP), glycerol, glycol, lauryl chloride, limonene, N-methylformamide, 2-phenylethanol, 3-phenyl-1-propanol, 3-phenyl-2-propen-1-ol, polyethylene glycol (MW 300-3000), polyoxyethylene sorbitan monoester, polypropylene glycol 425, primary alcohol (e.g., tridecanol), Procter & Gamble system: small polar solvent (1,2-propane diol, butanediol, C3-6 triol, or mixture thereof and a polar lipid compound selected from C16 or C18 monounsaturated alcohol, C16 or C18 branched saturated alcohol and mixture thereof), propylene glycol, squalene, triacetin, trichloroethanol, trifluoroethanol, trimethylene glycol, and xylene.

Additional PEs that may be used in the instant invention include: 1-N-dodecyl-2-pyrrolidone-5-carboxylic acid, octisalate, lauryl alcohol, propylene glycol monolaurate, lauroglycol, isopropyl myristate, triacetin, nonanol, oleyl alcohol, linoleyl alcohol, methyl laurate, glycerol monolaurate, glycerol monooleate, cetyl alcohol, stearyl alcohol, 1-dodecanol, oleyl alcohol, lactic acid, salicylic acid, bile salt, monoolein, 1-menthol, 2-n-nonyl-1,3-dioxolane, 1,3-dioxane and 1,3-dioxolane, alkanol (such as ethanol), ester such as ethyl acetate, and long chain (C7-C16) alkanes. See, for example, U.S. Pat. No. 5,118,676, U.S. Pat. No. 5,118,692, CA 2,610,708, NZ 522532, KR 100287626B, and CA 2,299,288 (all incorporated by reference).

Further PEs that may be used in the instant invention include: aliphatic thiol, alkyl N,N-dialkyl-substituted amino acetate, anise oil, anticholinergic agent pretreatment, ascaridole, biphasic group derivative, bisabolol, cardamom oil, 1-carvone, chenopodium (70% ascaridole), chenopodium oil, 1.8 Cineole (eucalyptol), cod liver oil (fatty acid extract), 4-decyloxazolidin-2-one, dicyclohexylmethylamine oxide, diethyl hexadecylphosphonate, diethyl hexadecylphosphoramidate, N,N-dimethyl dodecylamine-N-oxide, 4,4-dimethyl-2-undecyl-2-oxazoline, N-dodecanoyl-L-amino acid methyl ester, 1,3-dioxacycloalkane (SEPA), dithiothreitol, eucalyptol (cineole), eucalyptus oil, eugenol, herbal extract, lactam N-acetic acid ester, N-hydroxyethalaceamide, 2-hydroxy-3-oleoloxy-1-pyroglutamyloxypropane, menthol, menthone, morpholine derivative, N-oxide, nerolidol, octyl-β-D-(thio)glucopyranoside, oxazolidinone, piperazine derivative, polar lipid, polydimethylsiloxane, poly [2-(methylsulfinyl)ethyl acrylate), polyrotaxane, polyvinylbenzyldimethylalkylammonium chloride, poly(N-vinyl-N-methyl acetamide), prodrug, saline (skin hydration), sodium pyroglutaminate, terpene and azacyclo ring compound, Vitamin E (α-tocopherol), and Ylang-ylang oil.

See Osborne and Henke, Pharmaceutical Technology, pp. 58-66, November 1997; and Ahad et al., Expert Opin. Ther. Patents 19(7): 969-988, 2009.

It should be noted that, in this section and other sections of the instant specification, wherever a chemical compound is referred to using a specific trade name or trademark rather than a generic chemical name or formula, such as TRANSCUTOL®, it is intended that the chemical compound not be limited to any specific source or manufacture that is associated with the trade name or trademark. Rather, all equivalent diethylene glycol monoethyl ether or 2-(2-Ethoxyethoxy)ethanol compounds, including those identical in chemical nature but are provided by a different source or manufacturer (such as Carbitol, Carbitol cello solve, Dioxitol, Poly-solv DE, and Dowanol DE, Dowanol 17, Ektasolve DE, Solvolsol), are specifically included within the meaning of each referred to trade name or trademarked product.

### 4. Diseases or Conditions Treated

The subject topical formulation (e.g., gel, cream, lotion, gel-cream) can be formulated to include one or more cosmetic or pharmaceutically active ingredients for treating any skin or mucosal surface diseases or conditions.

In certain embodiments, the active ingredient of the topical formulation is effective to treat a skin or mucosal disease or condition, such as (without limitation): psoriasis, plaque psoriasis, nail psoriasis, psoriatic arthritis, hidradenitis suppurativa, alopecia areata/universalis, vitiligo, AKT keratosis (age spots), itch, or atopic dermatitis.

The term “skin or mucosal disease” or “skin or mucosal disorder” as used interchangeably herein, refers to skin or mucosal abnormalities other than injury wounds. In certain embodiments, the affected skin or mucosal surface may have induced a state of inflammation.

Thus in one embodiment, the skin or mucosal disease or disorder is an inflammatory skin/mucosal disorder, wherein the skin/mucosal surface is characterized by capillary dilatation, leukocytic infiltration, redness, heat, itch, and/or pain. Examples of such disorders include, but are not limited to, psoriasis, plaque psoriasis, nail psoriasis, psoriatic arthritis, pemphigus vulgaris, scleroderma, atopic dermatitis, alopecia areata/universalis, sarcoidosis, erythema nodosum, hidradenitis suppurativa, lichen planus, Sweet's syndrome, AKT keratosis (age spots), itch, and vitiligo. Additional disorders include chronic actinic dermatitis (also referred to as photosensitivity dermatitis/actinic reticuloid syndrome (PD/AR)), bullous pemphigoid, and alopecia areata.

In a specific embodiment, the skin, mucosal or nail disorder is one in which TNFα activity is detrimental, including skin, mucosal, and/or nail disorders and other disorders in which the presence of TNFα in a subject suffering from the disorder has been shown to be or is suspected of being either responsible for the pathophysiology of the disorder or a factor that contributes to a worsening of the disorder, e.g., psoriasis. Accordingly, in such disorder, inhibition of TNFα activity is expected to alleviate the symptoms and/or progression of the disorder. The use of any anti-TNFα antibodies, antibody portions, antibody mimetics, and other TNFα inhibitors in the treatment of specific skin disorders is performed in combination with another therapeutic agent known to be effective for treating the disorder.

For example, the subject topical formulation may be used to formulate one or more Disease Modifying Anti-Rheumatic Drug (DMARD) or a Non-steroidal Anti-inflammatory Drug (NSAID) or a steroid or any combination thereof. Preferred examples of a DMARD are hydroxychloroquine, leflunomide, methotrexate, parenteral gold, oral gold and sulfasalazine. Preferred examples of non-steroidal anti-inflammatory drug(s) also referred to as NSAIDS include drugs like ibuprofen. Other preferred combinations are corticosteroids including prednisolone.

Additional agents include: methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNFα or IL-1 (e.g., IRAK, NIK, IKK, p38 or MAP kinase inhibitors), IL-1β converting enzyme inhibitors, TNFα converting enzyme (TACE) inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g., soluble p55 or p75 TNF receptors and the derivatives p75 TNFRIgG (ENBREL™ and p55 TNFRIgG (Lenercept)), sIL-1RI, sIL-1RII, sIL-6R), anti-inflammatory cytokines (e.g., IL-4, IL-10, IL-12, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, human recombinant, tramadol HCl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline HCl, sulfadiazine, oxycodone HCl/acetaminophen, olopatadine HCl, misoprostol, naproxen sodium, omeprazole, cyclophosphamide, rituximab, IL-1 TRAP, MRA, CTLA4-IG, IL-18 BP, anti-IL-18, Anti-IL15, BIRB-796, SCIO-469, VX-702, AMG-548, VX-740, Roflumilast, IC-485, PSORIASIS C-801, and Mesopram.

Psoriasis refers to skin disorders associated with epidermal hyperplasia. Example of psoriasis include, but are not limited to, chronic plaque psoriasis, guttate psoriasis, inverse psoriasis, pustular psoriasis, psoriasis vulgaris, and erythrodermic psoriasis. Psoriasis can also be associated with other inflammatory disorders, including inflammatory bowel disease (IBD) and rheumatoid arthritis (RA).

Psoriasis is described as a skin inflammation (irritation and redness) characterized by frequent episodes of redness, itching, and thick, dry, silvery scales on the skin. In particular, lesions are formed which involve primary and secondary alterations in epidermal proliferation, inflammatory responses of the skin, and an expression of regulatory molecules such as lymphokines and inflammatory factors. Psoriatic skin is morphologically characterized by an increased turnover of epidermal cells, thickened epidermis, abnormal keratinization, inflammatory cell infiltrates into the epidermis and polymorphonuclear leukocyte and lymphocyte infiltration into the epidermis layer resulting in an increase in the basal cell cycle. Psoriasis often involves the nails, which frequently exhibit pitting, separation of the nail, thickening, and discoloration. Psoriasis is often associated with other inflammatory disorders, for example arthritis, including rheumatoid arthritis, inflammatory bowel disease (IBD), and Crohn's disease. Approximately one third of subjects with psoriasis also have psoriatic arthritis (PsA) which, as described above, causes stiffness, swelling of the joints, pain, and reduced range of motion (Greaves et al., (1995) N. Eng. J. Med., 332:581).

Evidence of psoriasis is most commonly seen, and is most usually treated, on the trunk, elbows, knees, scalp, skin folds, or fingernails, but it may affect any or all parts of the skin. Normally, it takes about a month for new skin cells to move up from the lower layers to the surface. In psoriasis, this process takes only a few days, resulting in a build-up of dead skin cells and formation of thick scales. Symptoms of psoriasis include: skin patches that are dry or red, covered with silvery scales, raised patches of skin, accompanied by red borders, that may crack and become painful, and that are usually located on the elbows, knees, trunk, scalp, and hands; skin lesions, including pustules, cracking of the skin, and skin redness; joint pain or aching which may be associated with of arthritis, e.g., psoriatic arthritis. Thus the topical formulation of the invention alleviates one or more symptoms of the disease.

In certain embodiments, one or more of the following therapeutics in a subject topical formulation are used to treat psoriasis: a topical corticosteroid, a vitamin D analog, a topical retinoid, or combinations thereof. In one embodiment, the topical formulation is administered in combination with or in the presence of one or more anti-TNFα biologics, such as a human, humanized, chimeric anti-TNFα antibodies, or antibody mimetics thereof.

Additional therapeutic agents which can also be combined for psoriasis treatment include: small molecule inhibitor of KDR (ABT-123), small molecule inhibitor of Tie-2, calcipotriene, clobetasol propionate, triamcinolone acetonide, halobetasol propionate, tazarotene, methotrexate, fluocinonide, betamethasone dipropionate, fluocinolone, acetonide, acitretin, betamethasone valerate, mometasone furoate, ketoconazole, pramoxine/fluocinolone, hydrocortisone valerate, flurandrenolide, urea, betamethasone, clobetasol propionate, fluticasone propionate, azithromycin, hydrocortisone, folic acid, desonide, coal tar, diflorasone diacetate, etanercept, folate, lactic acid, methoxsalen, HC-Bismut_Subgal_Znox_Resor, methylprednisolone acetate, prednisone, salicylic acid, halcinonide, anthralin, clocortolone pivalate, coal extract, coal tar/salicylic acid, coal tar/salicylic acid/sulfur, desoximetasone, diazepam, pimecrolimus, fluocinonide, mineral oil/castor oil, mineral oil/peanut oil, petroleum/isopropyl myristate, psoralen, salicylic acid, /tribromsalan, thimerosal/boric acid, celecoxib, infliximab, alefacept, efalizumab, tacrolimus, pimecrolimus, PUVA, UVB and other phototherapy, and sulfasalazine.

Psoriatic arthritis or psoriasis associated with the skin refers to chronic inflammatory arthritis which is associated with psoriasis, which is a common chronic skin condition that causes red patches on the body. About 1 in 20 individuals with psoriasis will develop arthritis along with the skin condition, and in about 75% of cases, psoriasis precedes the arthritis. PsA exhibits itself in a variety of ways, ranging from mild to severe arthritis, wherein the arthritis usually affects the fingers and the spine. When the spine is affected, the symptoms are similar to those of ankylosing spondylitis. PsA is sometimes associated with arthritis mutilans. Arthritis mutilans refers to a disorder which is characterized by excessive bone erosion resulting in a gross, erosive deformity which mutilates the joint.

Any of the APIs in the subject topical formulation effective to treat psoriasis, as described above, and are also effective to treat psoriatic arthritis and its associated skin disorders.

Other examples of agents which can be used to reduce or inhibit the symptoms of psoriatic arthritis, and which may be formulated in the instant topical formulation include: methotrexate; etanercept; rofecoxib; celecoxib; folic acid; sulfasalazine; naproxen; leflunomide; methylprednisolone acetate; indomethacin; hydroxychloroquine sulfate; sulindac; prednisone; betamethasone dipropionate; infliximab; methotrexate; folate; triamcinolone acetonide; diclofenac; dimethylsulfoxide; piroxicam; diclofenac sodium; ketoprofen; meloxicam; prednisone; methylprednisolone; nabumetone; tolmetin sodium; calcipotriene; cyclosporine; diclofenac; sodium/misoprostol; fluocinonide; glucosamine sulfate; gold sodium thiomalate; hydrocodone; bitartrate/apap; ibuprofen; risedronate sodium; sulfadiazine; thioguanine; valdecoxib; alefacept; and efalizumab.

Atopic dermatitis (AD, also referred to as eczema) is a hypersensitivity reaction (similar to an allergy) which occurs in the skin, causing chronic inflammation, and is categorized by scaly and itching plaques. Eczema patients often have a family history of allergic conditions like asthma, hay fever, or eczema. The inflammation causes the skin to become itchy and scaly. Chronic irritation and scratching can cause the skin to thicken and become leathery-textured. Exposure to environmental irritants can worsen symptoms, as can dryness of the skin, exposure to water, temperature changes, and stress. Symptoms often include intense itching, blisters with oozing and crusting, skin redness or inflammation around the blisters, rash, dry, leathery skin areas, raw areas of the skin from scratching, and ear discharges/bleeding. Thus the subject topical formulation alleviates at least one of the symptoms of atopic dermatitis, including that of moderate-to-severe AD.

The subject topical formulation for treating atopic dermatitis may include one or more APIs selected from: an IL-4R antagonist, an IL-1 antagonist (including, e.g., an IL-1 antagonist as set forth in U.S. Pat. No. 6,927,044), an IL-6 antagonist, an IL-6R antagonist (including, e.g., an anti-IL-6R antibody as set forth in U.S. Pat. No. 7,582,298), an IL-13 antagonist, a TNF antagonist, an IL-8 antagonist, an IL-9 antagonist, an IL-17 antagonist, an IL-5 antagonist, an IgE antagonist, a CD48 antagonist, an IL-31 antagonist (including, e.g., as set forth in U.S. Pat. No. 7,531,637), a thymic stromal lymphopoietin (TSLP) antagonist (including, e.g., as set forth in US 2011/027468), interferon-gamma (IFNγ) antibiotics, a calcineurin inhibitor, topical corticosteroids, tacrolimus, pimecrolimus, cyclosporine, azathioprine, methotrexate, cromolyn sodium, proteinase inhibitors, or combinations thereof. In certain embodiments, the topical formulation is administered to a subject in conjunction with a non-pharmaceutical therapy such as ultraviolet (UV) light therapy.

The term “TCS” or “topical corticosteroids” as used herein includes group I (weak), group II (moderately potent), group III (potent) and group IV (very potent) topical corticosteroids, according to the Anatomical Therapeutic Classification System of World Health Organization, based on their activity as compared to hydrocortisone. Group IV TCS (very potent) are up to 600 times as potent as hydrocortisone and include clobetasol propionate and halcinonide. Group III TCS (potent) are 50 to 100 times as potent as hydrocortisone and include, but are not limited to, betamethasone valerate, betamethasone dipropionate, diflucortolone valerate, hydrocortisone-17-butyrate, mometasone furoate, and methylprednisolone aceponate. Group II TCS (moderately potent) are 2 to 25 times as potent as hydrocortisone and include, but are not limited to, clobetasone butyrate, and triamcinolone acetonide. Group I TCS (mild) includes hydrocortisone.

Hidradenitis suppurativa refers to a skin disorder in which swollen, painful, inflamed lesions or lumps develop in the groin and sometimes under the arms and under the breasts. Hidradenitis suppurativa occurs when apocrine gland outlets become blocked by perspiration or are unable to drain normally because of incomplete gland development. Secretions trapped in the glands force perspiration and bacteria into surrounding tissue, causing subcutaneous induration, inflammation, and infection. Hidradenitis suppurativa is confined to areas of the body that contain apocrine glands. These areas are the axillae, areola of the nipple, groin, perineum, circumanal, and periumbilical regions.

Non-limiting examples of therapeutic agents for hidradenitis suppurativa and other skin disorders may include the following: antiseptic and antiperspirant agents (e.g., 6.25% aluminum chloride hexahydrate in absolute ethanol), anti-inflammatory or anti-antiandrogen therapy such as tetracycline, intralesional triamcinolone, finasteride, anti-TNF antibodies, adalimumab (HUMIRA®), CA2 (REMICADE®), PSORIASIS P 571, TNFR-Ig constructs, (p75 TNFRIgG (ENBREL™) and p55 TNFRIgG (LENERCEPT) inhibitors and PDE4 inhibitors, corticosteroids, for example, budenoside and dexamethasone, sulfasalazine, 5-aminosalicylic acid and olsalazine, and agents which interfere with synthesis or action of proinflammatory cytokines such as IL-1, for example, IL-1β converting enzyme inhibitors and IL-1ra, T cell signaling inhibitors, for example, tyrosine kinase inhibitors 6-mercaptopurines, IL-12, mesalamine, prednisone, azathioprine, mercaptopurine, infliximab, methylprednisolone sodium succinate, methotrexate, folate, tetracycline hydrochloride, fluocinonide, metronidazole, thimerosal/boric acid, ciprofloxacin hydrochloride, promethazine hydrochloride, hydrocortisone, balsalazide disodium, folic acid, levofloxacin, methylprednisolone, natalizumab and interferon-gamma.

Vitiligo refers to a skin condition in which there is loss of pigment from areas of skin resulting in irregular white patches with normal skin texture. Lesions characteristic of vitiligo appear as flat depigmented areas. The edges of the lesions are sharply defined but irregular. Frequently affected areas in subjects with vitiligo include the face, elbows and knees, hands and feet, and genitalia. In certain embodiments, the treatable vitiligo includes segmental vitiligo and non-segmental vitiligo, such as focal vitiligo, mucosal vitiligo, generalized vitiligo, vulgaris, and universal vitiligo.

Exemplary vitiligo topical formulations may include topical corticosteroids (such as prednisone, methylprednisolone and prednisolone), topical immunomodulators and psoralen phototherapy, mercaptopurine, alkylating agents such as cyclophosphamide, calcineurin inhibitors such as cyclosporine, sirolimus and tacrolimus, inhibitors of inosine monophosphate dehydrogenase (IMPDH) such as mycophenolate, mycophenolate mofetil, azathioprine, various antibodies, for example, antilymphocyte globulin (ALG), antithymocyte globulin (ATG), monoclonal anti-T-cell antibodies (OKT3), and irradiation.

In addition, the vitiligo topical formulations may be administered either in combination or adjunctively with systemic phototherapy (e.g., narrow-band UV-B phototherapy, 310-315 nm), psoralen photochemotherapy (PUVA: psoralens (e.g., 5-methoxypsoralen, 8-methoxypsoralen (0.1-0.3%), trimethylpsoralen) combined with UV-A light), excimer laser (308 nm) therapy. In some examples, topical tacrolimus (0.03-0.1%) ointment is combined with excimer laser therapy. Pimecrolimus (1%) cream may be combined with narrow-band UV-B treatment for vitiligo of the face. Vitamin D analogs (e.g., calcipotriol, tacalcitol) may be combined with narrow-band UV-B or PUVA treatment.

Further, the vitiligo topical formulations may be administered either in combination or adjunctively with an ophthalmic formulation of a drug such as an antihistamine, an antibiotic, an anti-inflammatory, an antiviral or a glaucoma medication for treating cases of vitiligo that primarily affect the eye or skin around the eye (such as the eyelids), and may be administered to or around the eye, for example in drops or ointments. When preparing these combination formulations, the vitiligo topical formulations may be combined with ophthalmic antibiotics (such as sulfacetamide, erythromycin, gentamicin, tobramycin, ciprofloxacin or ofloxacin); ophthalmic corticosteroids (such as prednisolone, fluorometholone or dexamethasone; ophthalmic non-steroidal anti-inflammatories (such as ibuprofen, diclofenac, ketorolac or flurbiprofen); ophthalmic antihistamines (such as livostin, patanol, cromolyn, alomide, or pheniramine); ophthalmic antiviral eye medications (such as triflurthymidine, adenine, arabinoside or idoxuridine); ophthalmic glaucoma medications (for example beta-blockers such as timolol, metipranolol, carteolol, betaxolol or levobunolol); ophthalmic prostaglandin analogues (such as latanoprost); ophthalmic cholinergic agonists (such as pilocarpine or carbachol); ophthalmic alpha agonists such as bromonidine or iopidine; ophthalmic carbonic anhydrase inhibitors (such as dorzolamide); and ophthalmic adenergic agonists (such as epinephrine or dipivefrin).

In general, the various cosmetic and therapeutic/pharmaceutical agents referenced herein can be used in accordance with their standard or common dosages, as specified in the prescribing information accompanying commercially available forms of the drugs (see also, the prescribing information in the 2006 Edition of The Physician's Desk Reference).

### EXAMPLES

The following examples are for illustration purpose only, and are not intended to be limiting. The specific embodiments described in the examples can be readily modified without departing from the spirit of the invention.

### Example 1 Production of Placebo Formulation—A

The following procedure was used to produce a batch of a placebo formulation (without active ingredients) of the subject invention. One of skill in the art can readily envision minor variations thereof without departing from the spirit of the invention. The ingredients of the formulation are listed below.

| | | | | Formulation |
|---|---|---|---|---|
| | | | Amount | Amount |
| Materials | CAS | Source | (Kg) | (wt %) |
| Mineral Oil, | 8042-47-5 | Spectrum | 1.200 | 15.0 |
| Light, NF | | Chemical | | |
| SEPINEO ™ | 38193-60-1, | Seppic S.A. | 0.240 | 3.0 |
| P 600 | 4390-04-9, | | | |
| | 9005-65-6 | | | |
| Transcutol ® HP, | 111-90-0 | Gattefosse SAS | 0.000 | 0.0 |
| USP/NF, EP | | | | |
| Imidurea | 39236-46-9 | Spectrum | 0.016 | 0.2 |
| (imidazolidinyl | | Chemical | | |
| urea), NF | | | | |
| Purified Water | | AbbVie, Inc. | 6.544 | 81.8 |
| Total Formulation | | | 8.000 | 100.0 |

1. Purge a 10 L jacketed reactor with nitrogen at a flow-rate of approximately 5 L/min for 30 min.
2. Charge 1.2 Kg of Mineral Oil, Light, NF into the 10 L reactor.
3. Charge 0.24 Kg of SEPINEO™ P 600 into the 10 L reactor. Agitate until homogeneous, then turn off agitation.
4. Charge 16 g Imidurea, NF to a pressure canister.
5. Charge 6.544 Kg of purified water into the appropriate pressure canister and agitate until all solids are dissolved.
6. Agitate the 10 L reactor at 100 rpm.
7. Setup a transfer line from the pressure canister to the 10 L reactor. Note: Agitation on the 10 L reactor was off.
8. Transfer the contents of the pressure canister to the 10 L reactor.
9. Start agitation of the 10 L reactor at 200 rpm. Mix for 6 minutes at 200 rpm (check particle size); mix for 12 minutes at 200 rpm (check particle size); and mix for 18 minutes at 250 rpm (check particle size). 10. Transfer the contents of the 10 L reactor to wide-mouth containers labelled as Placebo Formulation A.

### Example 2 Production of Placebo Formulation—B

The following procedure was used to produce a batch of a placebo formulation (without active ingredients) of the subject invention. One of skill in the art can readily envision minor variations thereof without departing from the spirit of the invention. The ingredients of the formulation are listed below.

| | | | | Formulation |
|---|---|---|---|---|
| | | | Amount | Amount |
| Materials | CAS | Source | (Kg) | (wt %) |
| Mineral Oil, | 8042-47-5 | Spectrum | 0.75 | 15.0 |
| Light, NF | | Chemical | | |
| SEPINEO ™ | 38193-60-1, | Seppic S.A. | 0.15 | 3.0 |
| P 600 | 4390-04-9, | | | |
| | 9005-65-6 | | | |
| Transcutol ® HP, | 111-90-0 | Gattefosse SAS | 0.50 | 10.0 |
| USP/NF, EP | | | | |
| Imidurea | 39236-46-9 | Spectrum | 0.01 | 0.2 |
| (imidazolidinyl | | Chemical | | |
| urea), NF | | | | |
| Purified Water | | AbbVie, Inc. | 3.59 | 71.8 |
| Total Formulation | | | 5.00 | 100.0 |

1. Purge a 10 L jacketed reactor with nitrogen at a flow-rate of approximately 5 L/min for 30 min.
2. Charge 0.75 Kg of Mineral Oil, Light, NF into the 10 L reactor.
3. Charge 0.15 Kg of SEPINEO™ P 600 into the 10 L reactor. Agitate until homogeneous, then turn off agitation.
4. Charge 10 g Imidurea, NF to a pressure canister.
5. Charge 0.50 Kg Transcutol® HP, USP/NF, EP to the pressure canister.
6. Charge 3.59 Kg of purified water into the appropriate pressure canister and agitate until solids are dissolved.
7. Setup a transfer line from the pressure canister to the 10 L reactor. Note: Agitation on the 10 L reactor is off.
8. Transfer the contents of the pressure canister to the 10 L reactor.
9. Start agitation of the 10 L reactor at 250 rpm. Sample the emulsion 12 and 25 minutes following the start of mixing and monitor particle size by optical microscopy.
10. Transfer the contents of the 10 L reactor to wide-mouth containers labelled as Placebo Formulation B.

### Example 3 Production of Topical Formulations for Four Compounds

A total of four topical formulations were prepared using two solid active pharmaceutical ingredients (sAPI)—Compound 2 and Compound 1—either with or without Transcutol® HP:
Compound 2 (1%) in Placebo Formulation A (no Transcutol® HP)
Compound 2 (1%) in Placebo Formulation B (10% Transcutol® HP)
Compound 1 (1%) in Placebo Formulation A (no Transcutol® HP)
Compound 1 (1%) in Placebo Formulation B (10% Transcutol® HP)
Cyclosporin A (0.1%) in Placebo Formulation B (10% Transcutol® HP)
Cyclosporin A (1%) in Placebo Formulation B (10% Transcutol® HP)
Metamethasone Dipropionate (0.01%) in Placebo Formulation B (10% Transcutol® HP)
Metamethasone Dipropionate (0.1%) in Placebo Formulation B (10% Transcutol® HP)
Metamethasone Dipropionate (1%) in Placebo Formulation B (10% Transcutol® HP)

For each formulation, the following general procedures were used:
1. Weigh 150 mg of sAPI and place on ground glass side of mixing plate.
2. Pre-weigh 14.85 g of the placebo formulation (with or without Transcutol® HP), prepared according to Example 1 or 2.
3. Place an equal volume portion of placebo on top of sAPI, and work the sAPI in with metal spatula, pay attention to minimizing particle size.
4. Once the particles are small enough, add an additional portion of placebo and mix/grind well.
5. Transfer the pre-mixed portion from the ground glass side of the slide to the smooth glass side of the slide.
6. Mix remaining placebo cream formulation with the sAPI-containing cream, blend well, and transfer the material to a vial for storage at room temperature.

Compound 1 and Compound 2 are both small molecule inhibitors for the JAK family kinases. Their structures are disclosed in WO 2011/068881 and WO 2011/068881, along with many other similar compounds, each of which can be used as API in the instant topical formulation.

Betamethasone Dipropionate (BMS DPP) is a “super high potency” corticosteroid with anti-inflammatory and immunosuppressive abilities. Although its exact mechanism of action is unclear, it is used to treat inflammatory skin conditions such as dermatitis, eczema and psoriasis.

Cyclosporin A (also known as ciclosporin, M.W. about 1202) is an immunosuppressant drug widely used in organ transplantation to prevent rejection. It is believed to reduce the activity of the immune system by interfering with the activity and growth of T cells. Cyclosporin is approved by the FDA to prevent and treat graft-versus-host (GVH) disease in bone-marrow transplantation and to prevent rejection of kidney, heart, and liver transplants. It is also approved in the US for the treatment of rheumatoid arthritis and psoriasis, as an ophthalmic emulsion for the treatment of dry eyes and as a treatment for persistent nummular keratitis following adenoviral keratoconjunctivitis. In addition to these indications, ciclosporin is also used in severe atopic dermatitis, Kimura disease, pyoderma gangrenosum, chronic autoimmune urticaria, acute systemic mastocytosis, and, infrequently, in rheumatoid arthritis and related diseases, particularly in severe cases. Cyclosporin has also been used to help treat patients with acute severe ulcerative colitis that do not respond to treatment with steroids; and as a treatment of posterior or intermediate uveitis with noninfective etiology. It is sometimes also prescribed in veterinary cases, particularly in extreme cases of immune-mediated hemolytic anemia.

### Example 4 Tests of Topical Formulations for Compound 1 and Compound 2

This example demonstrates that desired pharmacological effects can be achieved with a subject topical formulation, by optimizing the various parameters including drug load, solubility, formulation composition, and the presence of a permeation enhancer (PE). Various in vitro release methods were used to evaluate formulations by monitoring API flux through animal skin or artificial membrane.

Specifically, the impact of the various parameters on API flux was assessed by varying concentration of the API and the presence or absence of a permeation enhancer; by comparing in vitro flux to in vivo exposures; and by evaluating and comparing in vitro techniques using different synthetic membranes and hairless mouse skin.

It is suitable to use hairless mouse skin (as well as human cadaver skin) in ex vivo flux studies to understand the impact of formulation variables on API transport. However, in order to find an adequate formulation for human applications and to minimize the use of animal resources, a number of commercially available artificial membranes may be used to mimic skin permeation. Such membranes were used in a number of in vitro diffusion techniques in this example, including Franz Cell Assemblies and TRANSWELL® plates, to aid in the prediction of in vivo performance for topical formulations. Data presented herein also guides formulation development and provides insight into in vivo performance.

The placebo gel-cream formulation was prepared substantially according to the method of Example 1. Briefly, the bulk placebo formulation was prepared by charging a N₂ purged reactor with an appropriate stir paddle with mineral oil, light and SEPINEO™ P 600 brand hydro swelling droplet (HSD) polymer and mixed until uniform. Imidurea was dissolved in purified water, and the mixture added to the mineral oil, light and SEPINEO™ P 600 brand hydro swelling droplet (HSD) polymer blend. The mixture is agitated until homogeneous. The placebo formulation so prepared had about 15% (w/w) mineral oil, light, 3% (w/w) SEPINEO™ P 600 brand hydro swelling droplet (HSD) polymer, 0.2% imidurea, and the balance (about 81.8% (w/w)) is purified water.

Active drug formulation was prepared by adding the API and the various PE (such as the TRANSCUTOL® HP brand PE) manually to the above placebo formulation. The API and the PE were incorporated into the subject placebo gel-cream formulation by levigating with a mortar and pestle, or with the use of a spatula on an ointment slab.

A total of 8 formulations were prepared, with differences in the final concentration of the API, and the identity of the PE, if present. See table below.

Formulation Legends and Composition

| | 0.1% API | |
|---|---|---|
| Permeation Enhancers (PE) | (Compound 1) | 1% API (Compound 2) |
| No PE | Formulation C | Formulation A |
| 10% Transcutol ® HP (TC) | Formulation D | Formulation B |
| 10% Dimethyl isosorbide (DMI) | Formulation G | Formulation F |
| 10% Isopropyl myristate (IPM) | Formulation J | Formulation I |

Optical microscopy examination of the various formulations above (data not shown) shows that increasing the Transcutol® HP content solubilizes a greater amount of Compound 1. Thus maintaining a saturated solution with the presence of TRANSCUTOL HP™ is expected to increase in vitro flux and increase resultant skin exposures.

Diffusion assays were performed using Franz Cells and TRANSWELL® plates, with Tuffryn®, polycarbonate, or polyester membranes, as well as hairless mouse skin. HPLC was employed to analyze in vitro samples.

Specifically, for the in vitro release testing in Franz Cells, vertical diffusion cells were filled with receptor media (phosphate buffer with 2% BSA). The receptor media was then agitated, with the exception of setup and sampling. Temperature is maintained at 32.5±2.0° C. Artificial membrane was then placed on the top of the cell, along with a dosage wafer, the formulation to be evaluated, and a glass sealing cap. A formulation permeability test was run in triplicate. Samples were collected at various programmed intervals, and sampling was an automated process by the system. Fresh media was pushed into the bottom of the cell while simultaneous sampling occurs from the top of the cell. All wells were sampled in a serial manner with the time of each sample recorded by the instrument.

For in vitro release testing in TRANSWELL® plates, a two-compartment well system was used, with an internal well having a membrane as the formulation compartment, and an outer well filled with receptor media (phosphate buffer with 2% BSA). The TRANS WELL® plates were then agitated in a temperature-controlled incubator with shaker. Samples were manually pulled at pre-determined intervals with a pipette. New media was added to the receptor well after each sampling to make up the volume loss. The system parameters are provided in the table below.

System parameters and comparison

| | | | TRANSWELL ® | |
|---|---|---|---|---|
| System parameters | Franz Cells | | Plates | |
| membranes studied | Tuffryn ®/0.45 μm | | Polycarbonate/0.4 μm; | |
| | | | Polyester/0.4 μm | |
| Time Points (hrs) | 0.5, 1, 2, 3, 6, 9, | | 1, 2, 4, 6, & 8 | |
| | 12, 24, & 48 | | | |
| Vol. of formulation applied | 300 | μL | 100 | μL |
| Diffusion Area | 1.77 | cm² | 0.33 | cm² |
| Receptor Vol. | 7 | mL | 0.6 | mL |
| Vol. of sample pulled | 2.5 | mL | 0.1 | mL |
| Temperature | 32.5 ± 2.0° | C. | 32° | C. |

| | | |
|---|---|---|
| System | automatic | manual |

| | |
|---|---|
| Receptor Media | Phosphate Buffer (pH 7.4, with 2% BSA) |

Generally, flux is calculated by determining the amount of solute that diffuses across a membrane of known surface area per unit time. There are many ways to calculate this value. We measured solute concentration in known volumes of receiver media and calculated the total amount that diffused over the time course of the experiment. Therefore our calculation would be:

$$Flux = \frac{Q}{At}$$

Q=total amount diffused (μg)

A=membrane area (cm²)

t=time (hr)

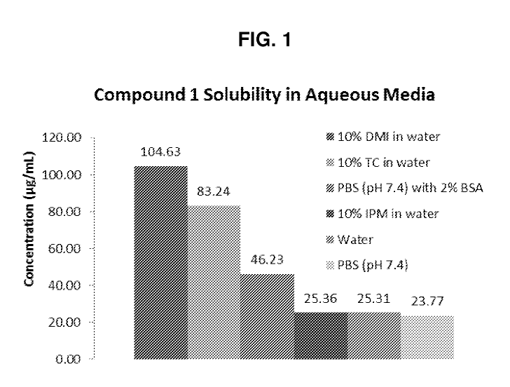

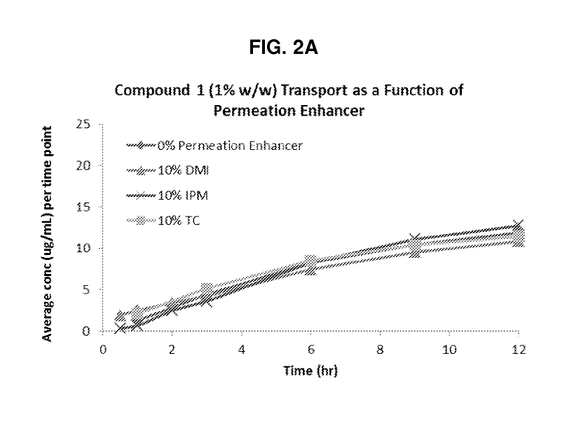

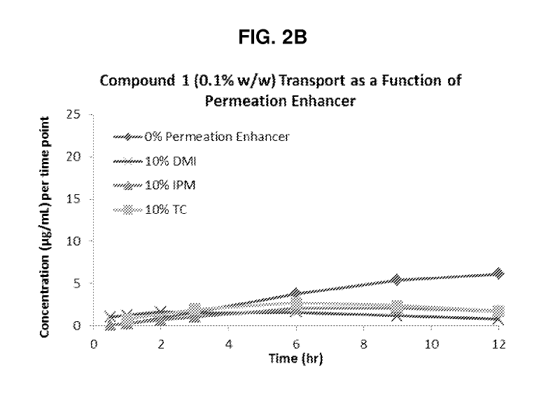

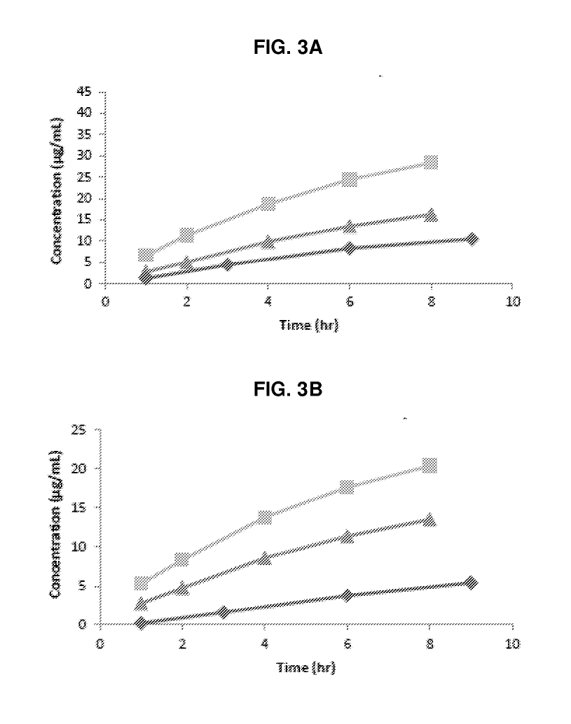

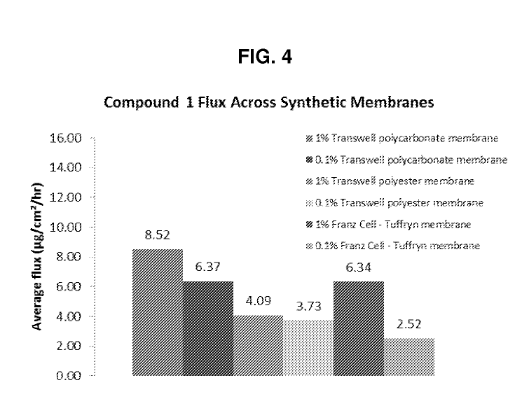

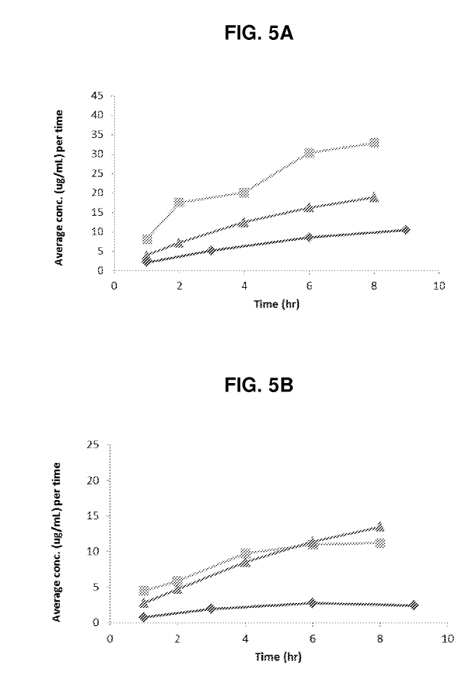

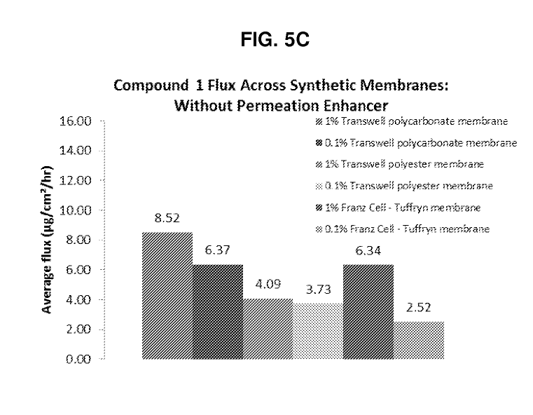

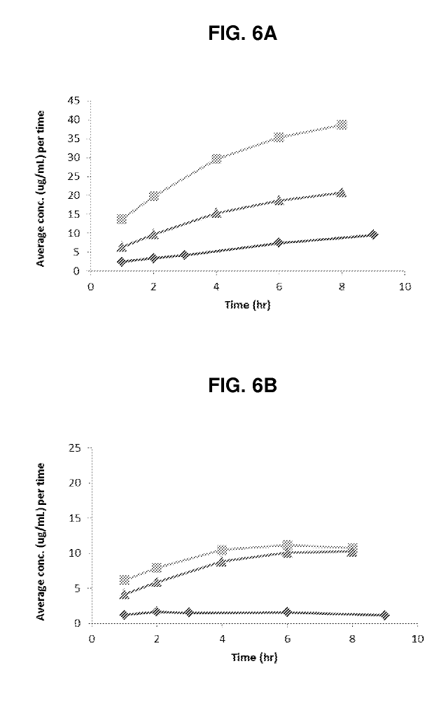

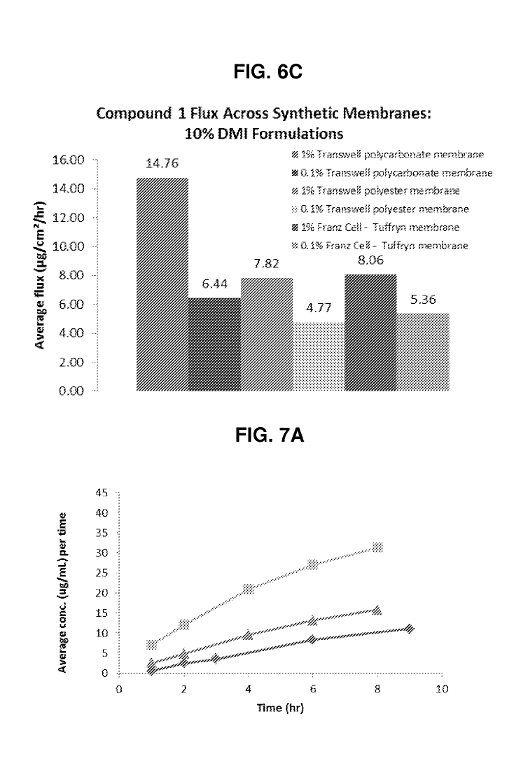

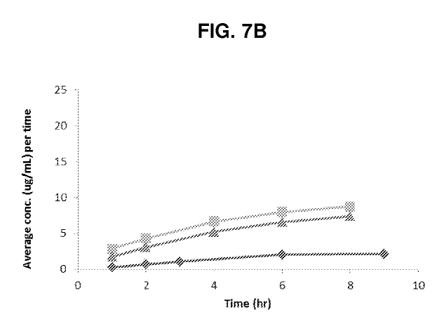

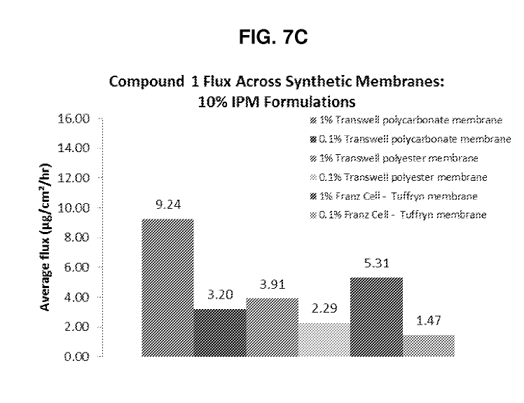

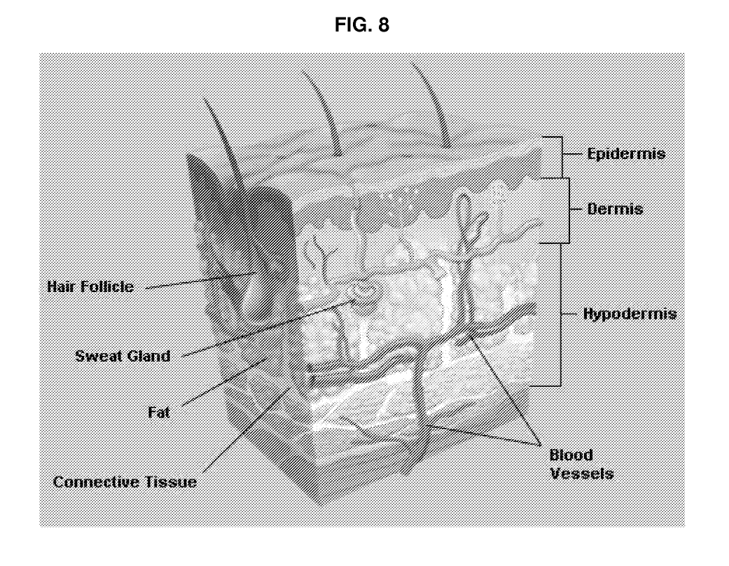

The results are shown in FIGS. 1-7C. The data shows that: in vitro, saturated systems with PE had greater flux than unsaturated/low dose formulations; and PE selection in saturated systems did not appear to substantially affect transport. In addition, synthetic membrane composition was shown to significantly impact in vitro diffusion. However, the relative rank order of formulation induced flux remained constant. A direct correlation between in vitro diffusion and in vivo PK data may be established for Compound 1 with the Franz Cell assembly.

Solubility measurements were conducted by equilibrating the API (i.e., Compound 1) in media by tumbling for NLT for 48 hrs, filtering with 0.45 μm filter, and followed by analysis by HPLC. The media evaluated include: water; PBS (ph7.4); PBS (pH7.4) with 2% BSA; 10% TRANSCUTOL® HP brand PE in water; 10% DMI in water; and 10% IPM in water. Results are shown in FIG. 1.

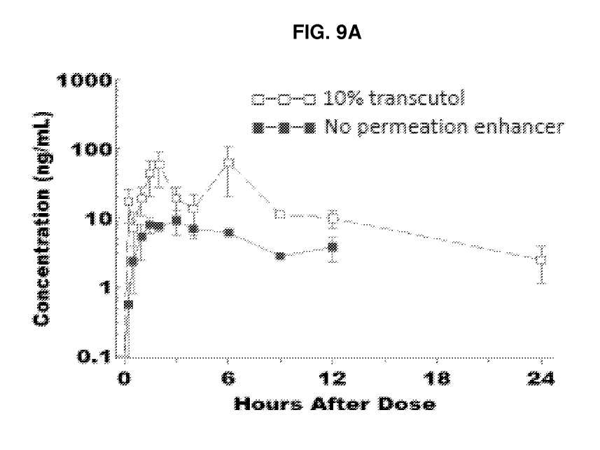

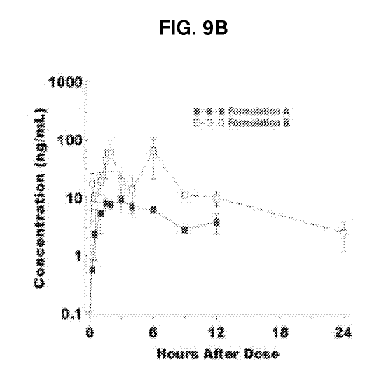

In order to assess the effect of the permeation enhancer on API delivery to the skin (topical delivery) and to the plasma (systamic delivery) in vivo, 177.8 mg of the subject topical formulation (177.8 μL of the 1% Compound 1 gel-cream, or about 1.778 mg of Compound 1) was applied to the backs of rats on a 4 cm² area of shaved skin. Plasma PK data was obtained during a period of about 24 hours following the topical application. See results in FIG. 9A, and the tables below.

| | | | | | Skin conc. | |
|---|---|---|---|---|---|---|
| | Cₘₐₓ | | AUC₀₋ₜ | | 24 hr | |
| | (ng/mL) | SEM | (ng*hr/mL) | SEM | (ng/g) | SEM |
| No | 11.4 | 2.7 | 62.4 | 6.6 | 5343 | 1566 |
| permeation | | | | | | |
| enhancer | | | | | | |
| 10% | 70.7 | 38.9 | 392 | 170 | 24962 | 4664 |
| Transcutol | | | | | | |
| HP ™ | | | | | | |

| | | | | Flux | |
|---|---|---|---|---|---|
| | | | Flux Franz | TRANSWELL ® | Flux |
| | Plasma | Skin conc. | Cells | plate | TRANSWELL ® |
| | AUC₀₋ₜ | 24 hr | Tuffryn | Polycarbonate | Plate polyester |
| | (ng * hr/mL | (ng/g) | membrane | membrane | membrane |
| Transcutol | 392 | 24962 | 7.64 | 11.46 | 5.42 |
| 10% | | | | | |
| No | 62.4 | 5343 | 6.34 | 8.52 | 4.09 |
| permeation | | | | | |
| enhancer | | | | | |
| Ratio | 6.28 | 4.67 | 1.20 | 1.35 | 1.33 |
| Transcutol | | | | | |
| HP ™/no | | | | | |
| enhancer | | | | | |

The data clearly shows that the presence of the PE increased API concentration both in the skin and in the plasma.

The same experiment was repeated for a topical formulation using Compound 2 as the API. The results are presented in the tables below. Also see FIG. 9B.

Variations of the above experimental procedures can be readily envisioned. For example, for experiments using hairless mouse skin or human skin, the dermis and/or the hypodermis layer(s) may be separated before the experiment, considering the fact that they are highly vascularized and there is high lymphatic drainage. Furthermore, API concentration in each human skin layer may be quantified, in order to assess the penetration of the topically delivered API to the different skin layers. The API concentration in such human/animal skin samples can be analyzed using different instruments. Finally, lowering API loads in the formulations, coupled with varying PE and concentration, may help to better stratify the formulations.

Compound 2 Topical (177.8 mg/animal) dosed in Formulation A

| | Dose | Time after | Plasma Conc. | Skin Conc. | Skin/Plasma |
|---|---|---|---|---|---|
| Rat # | (mg/kg) | dose (hr) | (ng/mL) | (ng/g) | Ratio |
| 1 | 4.70 | 24 | 0.00 | 2393 | nf |
| 2 | 4.45 | 24 | 0.00 | 5908 | nf |
| 3 | 4.47 | 24 | 0.00 | 7728 | nf |
| Mean | | | 0.00 | 5343 | |
| SD | | | 0.00 | 2715 | |
| SEM | | | 0.00 | 1566 | |

Compound 2 Topical 177.8 mg/animal dosed in Formulation B

| | Dose | Time after | Plasma Conc. | Skin Conc. | Skin/Plasma |
|---|---|---|---|---|---|
| Rat # | (mg/kg) | dose (hr) | (ng/mL) | (ng/g) | Ratio |
| 4 | 4.90 | 24 | 4.46 | 27131 | 6081 |
| 5 | 4.72 | 24 | 2.87 | 31734 | 11076 |
| 6 | 4.72 | 24 | 0.00 | 16020 | nf |
| Mean | | | 2.44 | 24962 | 8579 |
| SD | | | 2.26 | 8708 | 3531 |
| SEM | | | 1.31 | 4664 | |

### Example 5 Prophylactic Topical Treatment of FITC-Induced Contact Hypersensitivity (CHS) with Cyclosporin A (CsA) Topical Cream Formulations

Contact hypersensitivity (CHS) is an immunological response against a small chemical hapten. During the sensitization phase, dermal dendritic cells (DCs) and Langerhans cells take up the hapten peptide conjugate and process it and migrate to the draining lymph node, where they present the antigen (Ag) to naive T cells, leading to the formation of Ag-specific memory T cells. Subsequent contact with the same chemical results in the activation of memory T cells and the recruitment of inflammatory cells to the site of allergen exposure. This elicitation response is manifested as skin inflammation, which in a clinical context is called allergic contact dermatitis. One of the small chemical haptens is FITC. FITC-induced CHS is believed to be predominantly associated with the activation of Th2 cells. Upon challenging the skin with FITC in mice sensitized with FITC, it is believed that FITC-specific T cells are recruited to the skin and produce Th2 cytokines, including IL-4.

This experiment demonstrates that the subject formulation can efficiently deliver the relatively large Cyclosporin A (CsA) molecule (MW 1202) prophylactically across the skin prior to FITC epicutaneous application.

### Tested Formulations:

Cyclosporin A topical cream formulations with different concentrations (0.01, 0.1%, and 1% w/w) were prepared according to Example 3.

### Experimental Protocol

Briefly, on Day 0, ears of experimental animals were sensitized with FITC. On Day 6, ears of the treatment group animals (0.01%, 0.1%, and 1% treatment groups) were first provided topical treatment of the subject formulations one hour prior to the FITC challenge. Specifically, 20 μL total (10 μL inner ear, and 10 μL outer ear) of topical treatment were massaged all around the ear of each experimental animals prior to FITC epicutaneous application. Placebo group animals were similarly treated except that no CsA was included in the formulation. Naïve group animals received no treatment during the experiment. On Day 7, about 24 hours post FITC challenge, ear thickness (in mm) of all animals were measured.

### Test Results

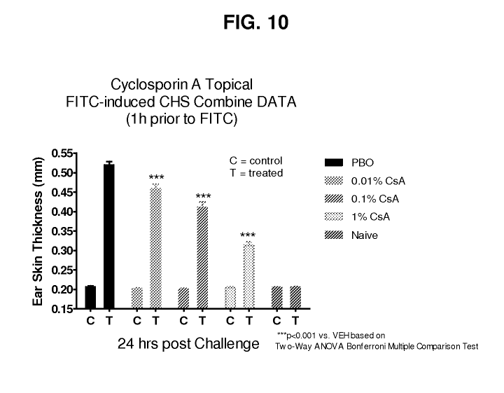

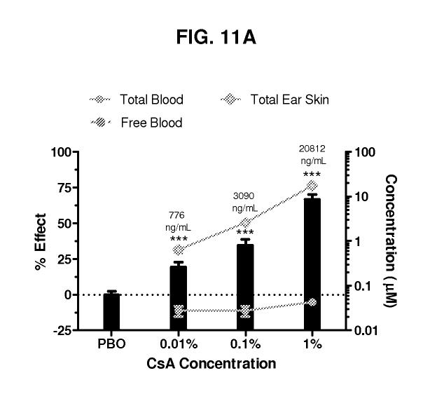

Two separate studies were conducted with CsA (0.01%, 0.1% and 1%) topical formulations prophylactically applied at 1 hour prior to FITC challenge. In Study 1, CsA (0.01%, 0.1% and 1%) prophylactically applied at 1 hr prior to FITC challenge reduced ear skin thickness in a dose dependent manner with effects of 19%, 32% and 58%, respectively. Similarly, in Study 2, CsA (0.01%, 0.1% and 1%) prophylactically applied at 1 hr prior to FITC challenge reduced ear skin thickness in a dose dependent manner with effects of 20%, 37% and 75%, respectively. Thus, after combining data from Study 1 and Study 2, CsA (0.01%, 0.1% and 1%) prophylactically applied at 1 hr prior to FITC challenge reduced ear skin thickness with effects of 19%, 34% and 67%, respectively. FIG. 10 shows the combined data from two studies.

Notably, statistical analysis shows that CsA is effective even at the lowest dose tested (0.01%), in that animals prophylactically treated by 0.01% CsA about 1 hr prior to FITC challenge have a small, but statistically significant (p<0.001) drop in ear skin thickness increase compared to control animals. At the highest does tested (1%), about 50% of the ear skin thickness increase in control animals was blocked. See FIG. 10.

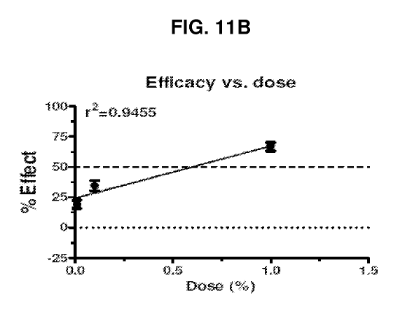

Pharmacokinetic (PK) analysis was performed for the three dosing groups (0.01%, 0.1%, and 1%) vs. the placebo group. It was found that the total blood concentration of CsA was relatively comparable among the three treatment groups (e.g., about 2-3 fold increase between the 0.01% and 1% groups) despite the 100-fold concentration difference in the topical formulations used. In contrast, the total ear skin concentration for CsA was about 776 ng/mL in the 0.01% treatment group, vs. the 20,812 ng/mL in the 1% treatment group (a 27-fold increase). See FIG. 11A. The data suggests that the subject formulation is not only effective for topical delivery, but also has minimual “leakage” of the API into central circulation. The data further suggests that the subject formulation topically delivers API to achieve therapeutic efficacy in a dose dependent manner. See the linear relationship between does and efficacy in FIG. 11B.

### Example 6 Topical Treatment in FITC-Induced Contact Hypersensitivity (CHS) with Cyclosporin A (CsA) Topical Cream Formulations

This experiment was designed to address the question of whether the skin barrier need to be defective for large molecule such as CsA to penetrate the skin more sufficiently, or, in other words, whether topical treatment using relatively large molecules such as CsA can achieve more effective skin barrier penetration if the formulation is applied after the skin has first been perturbed (e.g., by FITC epicutaneous application).

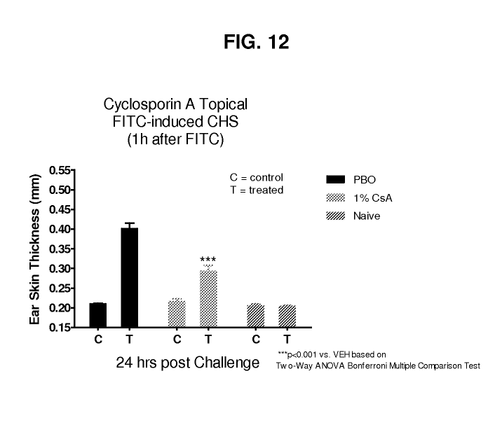

The experimental set up is essentially the same as that in Example 5, except that the order of FITC challenge and topical treatment on Day 6 was reversed (that is, FITC challenge was performed one hour prior to topical treatment on Day 6). The result is shown in FIG. 12—at the does tested (1%), about 57% of the ear skin thickness increase in control animals was blocked, which result was comparable to that using the same (1%) topical formulation applied prior to FITC challenge.

To further investigate whether this 1 hr post FITC challenge treatment window is too early for CD4⁺ T cells to infiltrate or migrate into the tissue to justify a therapeutic effect, the 1 hr delayed treatment window was increased to 6 hours post FITC challenge in a follow-up experiment.

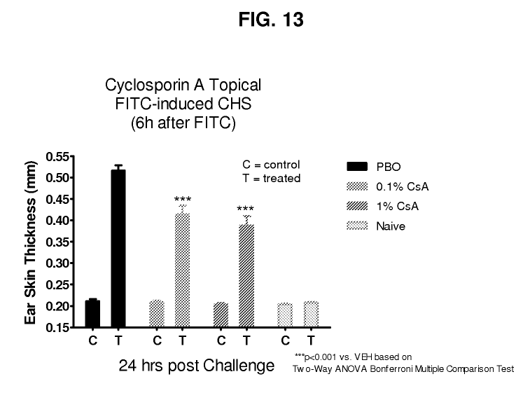

The results in FIG. 13 show that: (1) CsA (0.1% and 1%) applied at 6 hrs post FITC challenge reduced ear skin thickness at 35% and 44%, respectively; and, (2) CsA at 1% applied at 1 hr or 6 hrs post FITC challenge were somewhat comparable in reducing ear thickness, 57% vs. 44%, respectively.

### Example 7 Prophylactic Topical Treatment of FITC-Induced Contact Hypersensitivity (CHS) with Betamethasone Dipropionate (BMS DPP) Topical Cream Formulations

Similar to Example 5, this example demonstrates that BMS DPP in the subject formulation can be used to prophylactically treat FITC-induced CHS.

Betamethasone Dipropionate (BMS DPP) topical cream formulations with different concentrations (0.005%, 0.01%, 0.1%, and 1% w/w) were prepared according to Example 3. Essentially the same experimental protocol as that used in Example 5 was used, except that the subject formulation with BMS DPP (instead of CsA) was used.

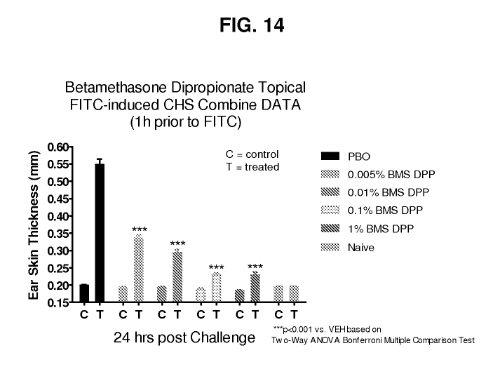

Two studies were conducted with BMS DPP (0.005%, 0.01%, 0.1% and 1%) topical formulations applied at 1 hour prior to FITC challenge. In Study 1, BMS DPP (0.01%, 0.1% and 1%) applied at 1 hr prior to FITC challenge reduced ear skin thickness at 77%, 93% and 95%, respectively. In Study 2, BMS DPP (0.005%, 0.01% and 0.1%) applied at 1 hr prior to FITC challenge reduced ear skin thickness at 61%, 73% and 93%, respectively. In the second study, a lower concentration of 0.005% topical was used instead of repeating the same concentration, because both 0.1% and 1% effectively blocked ear skin thickness induced by FITC in the first study. After combining data from Study 1 and Study 2, BMS DPP (0.005%, 0.01%, 0.1% and 1%) applied at 1 hr prior to FITC challenge reduced ear skin thickness with effects of 61%, 75%, 93%, and 95%, respectively. The results are shown in FIG. 14.

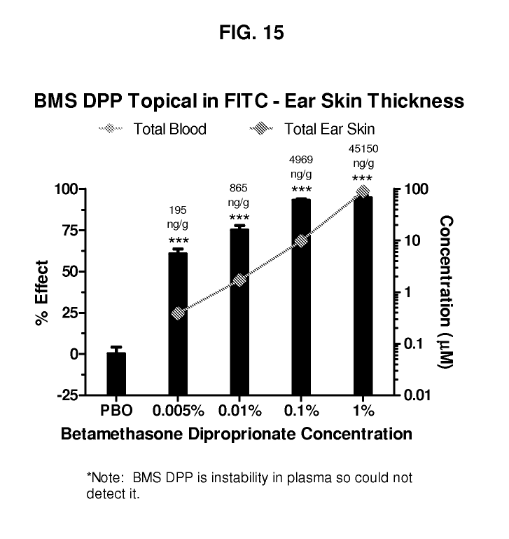

The PK study data is shown in FIG. 15. Note that, due to the fact that BMS DPP is unstable in the plasma, the total blood concentration of BMS DPP cannot be determined.

### Example 8 Topical Treatment of FITC-Induced Contact Hypersensitivity (CHS) with Betamethasone Dipropionate (BMS DPP) Topical Cream Formulations

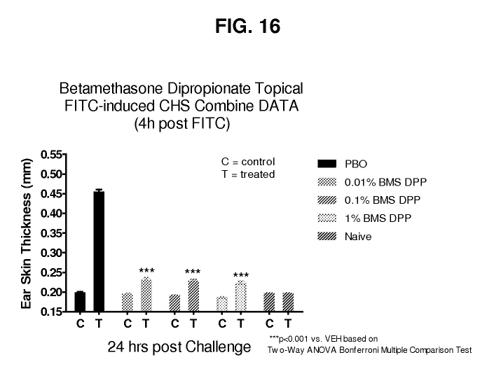

Similarly, the same experiment as in Example 6 was conducted to test whether a 4-hour delay in BMS DPP treatment after initial FITC challenge is still effective. The experimental set up is essentially the same as that in Example 7 except that the BMS DPP treatment on Day 6 was administered 6 hours after FITC challenge, and the results are in FIG. 16.

Again, two studies were conducted with BMS DPP topical formulations applied at 4 hour post FITC challenge. In Study 1, BMS DPP (0.1% and 1%) applied at 4 hrs post FITC challenge reduced ear skin thickness at 95% and 96%, respectively. In Study 2, BMS DPP (0.01% and 0.1%) applied at 4 hrs post FITC challenge reduced ear skin thickness at 92% and 96%, respectively. In the second study, a lower concentration of 0.01% topical was used instead of repeating the same concentration, because both 0.1% and 1% practically blocked ear skin thickness induced by FITC in the first study. The second study showed that even at 0.01%, BMS DPP effectively blocked ear skin thickness induced by FITC when applied at 4 hrs post FITC challenge. Combining data from Study 1 and Study 2, BMS DPP (0.01%, 0.1% and 1%) in the subject formulation applied at 4 hrs post FITC challenge reduced ear skin thickness with effects of 92%, 95%, and 96%, respectively.

Each patent, patent application, publication, text and literature article/report cited or indicated herein is hereby expressly incorporated by reference in its entirety.

While the invention has been described in terms of various specific and illustrated embodiments, the skilled artisan will appreciate that various modifications, substitutions, omissions, and changes may be made without departing from the spirit thereof.

1. A method for producing a formulation for topical application to skin or mucosal surface, the method comprising:
(1) mixing an oil phase with an emulsifying agent to form a mixture, and agitating the mixture until homogeneous;
(2) adding an aqueous phase and continuing to agitate to form a placebo formulation;
(3) adding a solid form active ingredient to the placebo formulation, and mixing until homogeneous;
wherein the method does not comprise a step that subjects the solid form active ingredient to a temperature higher than 35° C.

2. The method of claim 1, wherein the solid form active ingredient is not subjected to a temperature higher than 15-30° C., or is subjected to a temperature between 15-30° C.

3. The method of claim 2, wherein the emulsifying agent is also a thickener, a stabilizing agent, or both.

4. The method of claim 1, further comprising independently adding a thickener, a stabilizing agent, or both a thickener and a stabilizing agent, in step (1) or step (2).

5. The method of claim 4, wherein the thickener does not require heating, or require heating above 35° C., for use in a topical formulation.

6. The method of claim 4, wherein the thickener comprises one or more of: silicon dioxide; xanthan gum; high molecular weight, crosslinked, acrylic acid-based polymers; polyvinyl alcohol; agarose; alginate; carrageenan; guar gum; cellulose derivative; methylcellulose; sodium carboxymethylcellulose; hydroxypropyl cellulose; hydroxypropyl methylcellulose; hydroxyethyl cellulose; and povidone.

7. The method of claim 4, wherein the stabilizing agent comprises: lauryl glucoside, decyl glucoside sodium cocoamphoacetate, polyglyceryl 3-methylglucose distearate, cetearyl glucoside, inulin lauryl carbamate, lecithin and its derivatives, purified phospholipids, polysorbate 80, sorbitan monooleate, or mixtures thereof.

8. The method of claim 4, wherein the stabilizing agent comprises:
polyoxyethylene 20 sorbitan monooleate; polyoxyethylene 20 sorbitan monolaurate;
polyoxyethylene 4 sorbitan monolaurate; polyoxyethylene 20 sorbitan monopalmitate;
polyoxyethylene 5 sorbitan monooleate; polyoxyethylene 20 sorbitan trioleate;
sorbitan monolaurate; sorbitan monooleate; sorbitan trioleate; polyoxyl 35 castor oil;
or mixtures thereof.

9. The method of claim 4, wherein the stabilizing agent comprises a polyoxyethylene sorbitan fatty acid ester.

10. The method of claim 4, wherein the stabilizing agent comprises a polyoxyethylene castor oil derivative.

11. The method of claim 4, wherein the stabilizing agent comprises a pharmaceutically acceptable liquid stabilizer that can be processed at ambient conditions for use in topical formulation.

12. The method of claim 11, wherein the ambient conditions are at a temperature between about 15-30° C.

13. The method of claim 1, further comprising adding a preservative to the aqueous phase before step (2).

14. The method of claim 1, further comprising adding a permeation enhancer to the mixture of step (1), between steps (1) and (2), to the aqueous phase of step (2), or between steps (2) and (3).

15. The method of claim 1, wherein the formulation is an oil-in-water emulsion, a water-in-oil emulsion, a lotion, a cream, a gel, or a gel-cream.

16. The method of claim 1, wherein the oil phase is about 1-30% (w/w), about 10-20% (w/w), or about 15% (w/w) of the formulation.

17. The method of claim 1, wherein the emulsifying agent is about 1-5% (w/w), or about 3% of the formulation.

18. The method of claim 13, wherein the preservative is about 0.001-2% (w/w), about 0.01-1% (w/w), about 0.1-0.5% (w/w), or about 0.2% of the formulation.

19. The method of claim 14, wherein the permeation enhancer is less than 15% (w/w), or about 1-10% of the formulation.

20. The method of claim 1, wherein the solid form active ingredient is about 0.01-10% (w/w), about 0.05-5% (w/w), about 0.1-1% (w/w), or about 0.5-1% (w/w) of the formulation.

21. The method of claim 1, wherein the oil phase comprises a pharmaceutically acceptable oil that can be processed at ambient conditions for use in topical formulation.

22. The method of claim 1, wherein the oil phase comprises: cod liver oil, light mineral oil, heavy mineral oil, shark liver oil, caprylic/capric triglyceride, vegetable oil, or mixtures thereof, wherein the vegetable oil comprises: castor oil, corn oil, canola oil, cottonseed oil, peanut oil, sesame oil, or soybean oil.

23. The method of claim 1, wherein the emulsifying agent comprises: sodium lauryl sulfate, a non-ionic emulsifier, or a SEPINEO™ P 600-type hydro swelling droplet polymer in an inverse emulsion.

24. The method of claim 23, wherein the inverse emulsion is acrylamide/sodium acryloyldimethyl taurate copolymer/isohexadecane/polysorbate 80, or SEPINEO™ P 600 brand HSD polymer.

25. The method of claim 13, wherein the preservative comprises: sodium benzoate, benzoic acid, sorbic acid, benzethonium chloride, benzalkonium chloride, bronopol, methylparaben, ethylparaben, propylparaben, butylparaben, thiomerosal, sodium propionate, chlorhexidine, chlorobutanol, chlorocresol, cresol, imidazolidinyl urea, diazolidinyl urea, phenol, phenylmercuric salts, potassium sorbate, propylene glycol, or mixtures thereof.

26. The method of claim 14, wherein the permeation enhancer comprises: dimethyl isosorbide, isopropyl myristate, diethylene glycol monoethyl ether, ethyl alcohol, ethyl oleate, isostearyl alcohol, oleyl alcohol, polyethylene glycol, N-methyl-2-pyrrolidone, dimethyl sulfoxide, or propylene carbonate.

27. The method of claim 1, wherein the aqueous phase is purified water.

28. The method of claim 1, wherein the solid form active ingredient is a cosmetic or pharmaceutical active ingredient.

29. The method of claim 25, wherein the solid form active ingredient is: Compound 1, Compound 2, Cyclosporin A or betamethasone dipropionate; or wherein the solid form active ingredient is effective to treat psoriasis, plaque psoriasis, nail psoriasis, psoriatic arthritis, hidradenitis suppurativa, alopecia areata/universalis, vitiligo, AKT keratosis (age spots), itch, or atopic dermatitis.

30. A cosmetic or pharmaceutical topical formulation for applying to skin or a mucosal surface, said topical formulation comprises:
(1) about 1-30% (w/w) oil;
(2) about 1-5% (w/w) emulsifying agent;
(3) about 1-15% (w/w) permeation enhancer;
(4) about 0.001-2% (w/w) preservative;
(5) about 65-90% (w/w) aqueous phase; and
(6) about 0.01-10% (w/w) cosmetic or pharmaceutical active ingredient.

31. The formulation of claim 30, wherein said topical formulation comprises:
(1) about 10-20% (w/w) oil;
(2) about 3% (w/w) emulsifying agent;
(3) about 10% (w/w) permeation enhancer;
(4) about 0.01-1% (w/w) preservative;
(5) about 82% (w/w) aqueous phase; and
(6) about 0.05-5%, (w/w) cosmetic or pharmaceutical active ingredient.

32. The formulation of claim 31, wherein said topical formulation comprises:
(1) about 0.1-0.5%, (w/w) preservative; and
(2) about 0.1-1%, (w/w) cosmetic or pharmaceutical active ingredient.

33. The topical formulation of claim 30, which is an oil-in-water formulation, a cream, a lotion, a gel, or a gel-cream.

34. The topical formulation of claim 30, wherein the mucosal surface is mucosa in the mouth, vaginal mucosal surface, mucosa of the eye.

35. The topical formulation of claim 30, wherein the active ingredient is: Compound 1, Compound 2; or wherein the active ingredient is effective to treat psoriasis, plaque psoriasis, nail psoriasis, psoriatic arthritis, hidradenitis suppurativa, alopecia areata/universalis, vitiligo, AKT keratosis (age spots), itch, or atopic dermatitis.

36. The topical formulation of claim 35, wherein the active ingredient is Compound 1.

37. The topical formulation of claim 35, wherein the active ingredient is Compound 2.

38. A method for treating a skin or mucosal surface lesion, the method comprising applying to the lesion a topical formulation of claim 30.

39. The method of claim 38, wherein the lesion is caused by psoriasis, plaque psoriasis, nail psoriasis, psoriatic arthritis, hidradenitis suppurativa, alopecia areata/universalis, vitiligo, AKT keratosis (age spots), itch, or atopic dermatitis.
